(19) United States
(12) Patent Application Publication
(10) Pub. No.: US 2021/0198315 A1
(43) Pub. Date: Jul. 1, 2021

(54) PROCESS FOR SYNTHESIS OF PEPTIDE COMPOUNDS

(71) Applicant: NAUREX, INC, Madison, NJ (US)

(72) Inventor: M. Amin KHAN, Evanston, IL (US)

(73) Assignee: Naurex, Inc, Madison, NJ (US)

(21) Appl. No.: 16/074,307

(22) PCT Filed: Jan. 31, 2017
(86) PCT No.: PCT/US2017/015851

§ 371 Date: Jul. 31, 2018

Related U.S. Application Data
(60) Provisional application No. 62/289,655, filed on Feb. 1, 2016.

(51) Int. Cl. C07K 5/107; C07C 271/66

(52) U.S. Cl. CPC C07K 5/1016; C07C 271/66

(57) ABSTRACT

Disclosed is a new process for preparing dipyrrolidine peptide compounds such as, for example, GLYX-13. Advantageously, the process may be industrially scalable and cost-effective and use less toxic reagents and/or solvents. Further, the process may be used to prepare peptide compounds having improved purity.

This application claims priority to and the benefit of U.S. Provisional Patent Application No. 62/289,655, filed on Feb. 1, 2016, the entire disclosure of which is incorporated herein by this reference.

### BACKGROUND

An N-methyl-D-aspartate (NMDA) receptor is a postsynaptic, ionotropic receptor that is responsive to, inter alia, the excitatory amino acids glutamate and glycine and the synthetic compound NMDA. The NMDA receptor (NMDAR) appears to controls the flow of both divalent and monovalent ions into the postsynaptic neural cell through a receptor associated channel and has drawn particular interest since it appears to be involved in a broad spectrum of CNS disorders. The NMDAR has been implicated, for example, in neurodegenerative disorders including stroke-related brain cell death, convulsive disorders, and learning and memory. NMDAR also plays a central role in modulating normal synaptic transmission, synaptic plasticity, and excitotoxicity in the central nervous system. The NMDAR is further involved in Long-Term Potentiation (LTP), which is the persistent strengthening of neuronal connections that underlie learning and memory The NMDAR has been associated with other disorders ranging from hypoglycemia and cardiac arrest to epilepsy. In addition, there are preliminary reports indicating involvement of NMDA receptors in the chronic neurodegeneration of Huntington's, Parkinson's, and Alzheimer's diseases. Activation of the NMDA receptor has been shown to be responsible for post-stroke convulsions, and, in certain models of epilepsy, activation of the NMDA receptor has been shown to be necessary for the generation of seizures. In addition, certain properties of NMDA receptors suggest that they may be involved in the information-processing in the brain that underlies consciousness itself. Further, NMDA receptors have also been implicated in certain types of spatial learning.

In view of the association of NMDAR with various disorders and diseases, NMDA-modulating small molecule agonist and antagonist compounds have been developed for therapeutic use. NMDA receptor compounds may exert dual (agonist/antagonist) effect on the NMDA receptor through the allosteric sites. These compounds are typically termed “partial agonists”. In the presence of the principal site ligand, a partial agonist will displace some of the ligand and thus decrease Ca⁺⁺ flow through the receptor. In the absence of the principal site ligand or in the presence of a lowered level of the principal site ligand, the partial agonist acts to increase Ca⁺⁺ flow through the receptor channel.

Recently, an improved partial agonist of NMDAR with the following structure has been reported:

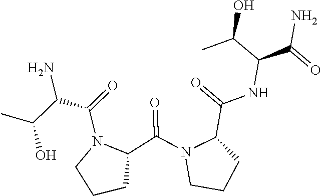

However, a need exists for improved GLYX-13 synthetic methods that, for example, minimize the use of costly and/or toxic reagents, eliminate cumbersome purification steps, are more efficient, result in higher purity GLYX-13, and can be utilized in large-scale industrial production of GLYX-13.

### SUMMARY

Disclosed is a new process for preparing dipyrrolidine peptide compounds such as, for example, GLYX-13. Advantageously, the process may be industrially scalable and cost-effective and use less toxic reagents and/or solvents. Further, the process may be used to prepare peptide compounds having improved purity.

In one aspect, a process for synthesizing a dipyrrolidine peptide compound or a pharmaceutically acceptable salt, stereoisomer, metabolite, or hydrate thereof is provided. The process comprises the steps:
a) contacting a compound of Formula III:

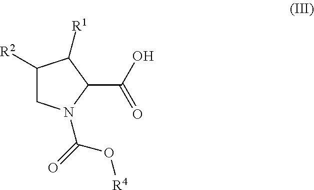

with an activating reagent and a compound of Formula II:

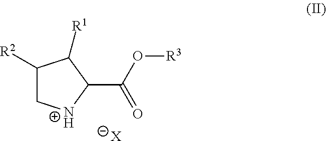

to produce a compound of Formula IV:

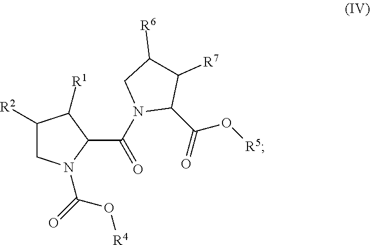

b) contacting the compound of Formula IV with a reagent capable of effecting hydrolysis to produce a compound of Formula V:

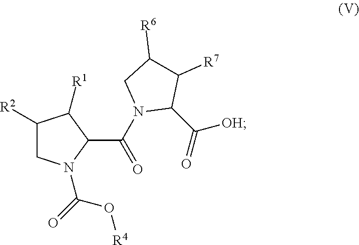

c) contacting the compound of Formula V with an activating reagent and a compound of Formula VIII:

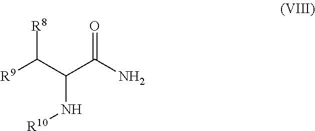

to produce a compound of Formula IX:

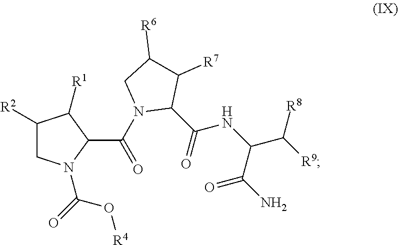

wherein:

R¹, R², R³, R⁴, R⁵, R⁶, R⁷, R⁸, R⁹, R¹⁰, R¹¹, and R¹² are as defined below. In some embodiments, step (a) is carried out at a temperature between about −10° C. and about 10° C. In some embodiments, step (b) is carried out at a temperature between about 15° C. and about 30° C. In some embodiments, step (c) is carried out at a temperature between about 0° C. and about 30° C.

In some embodiments, the process further comprising the steps:
d) contacting the compound of Formula IX with a carbamate-cleaving reagent to produce a compound of Formula XI:

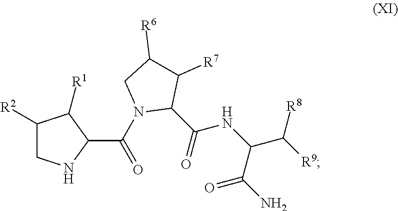

e) contacting a compound of Formula X:

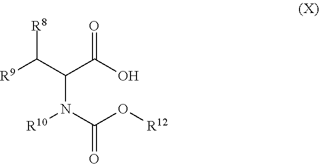

with an activating reagent and the compound of Formula XI to produce a compound of Formula XII:

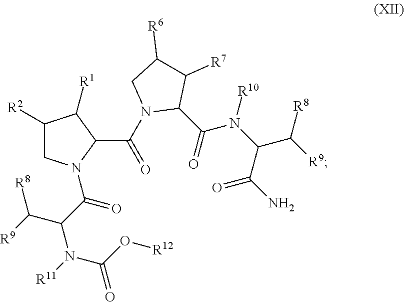

f) contacting the compound of Formula XII with a carbamate-cleaving reagent to produce a compound of Formula XIII:

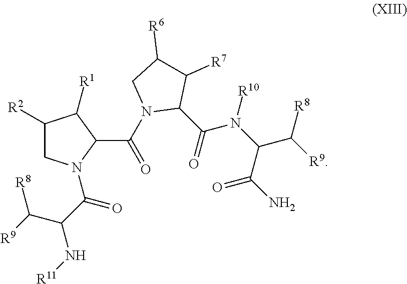

In some embodiments, step (d) is carried out at a temperature between about 15° C. and about 30° C. In some embodiments, step (e) is carried out at a temperature between about −10° C. and about 30° C. In some embodiments, step (f) is carried out at a temperature between about 15° C. and about 30° C. In certain embodiments, the compound of Formula X is produced by contacting a compound of Formula VI:

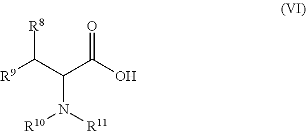

with an activated carbonyl compound. In some embodiments, the compound of Formula VIII is produced by the steps:
g) contacting a compound represented by Formula VI:

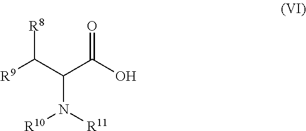

with an activating reagent to form a compound represented by Formula VII:

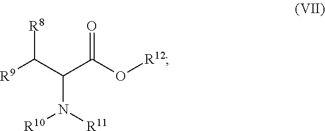

and
h) contacting the compound of Formula VII with an amine to produce the compound of Formula VIII. In some embodiments, step (g) is carried out at a temperature between about −10° C. and about 100° C. In some embodiments, step (h) is carried out at a temperature between about 15° C. and about 30° C.

In some cases, the compound of Formula II is produced by contacting a compound of Formula I:

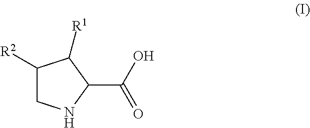

with an activating reagent and an alcohol. In some embodiments, producing the compound of Formula II is carried out at a temperature of between about 0° C. to about 100° C. In other embodiments, producing the compound of Formula II is carried out at a temperature of between about 0° C. to about 5° C.

In another aspect, a process for preparing a dipyrrolidine peptide compound or a pharmaceutically acceptable salt, stereoisomer, metabolite, or hydrate thereof is provided. The process comprises the steps:
a) contacting a compound of Formula IX:

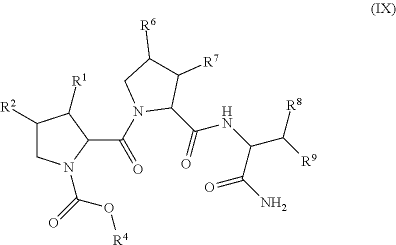

with a carbamate-cleaving reagent to produce a compound of Formula XI:

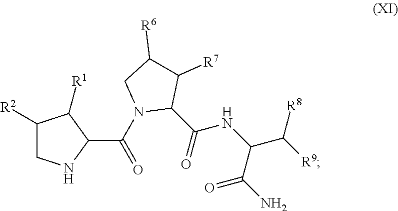

b) contacting a compound of Formula X:

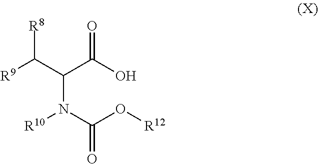

with an activating reagent and the compound of Formula XI in the presence of at least one solvent to produce a compound of Formula XII:

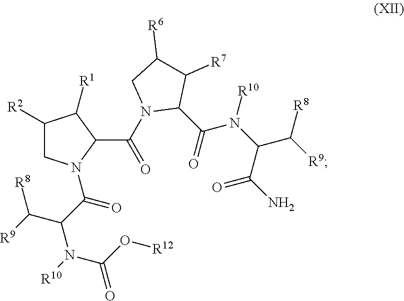

c) contacting the compound of Formula XII with a carbamate-cleaving reagent to produce a compound of Formula XIII:

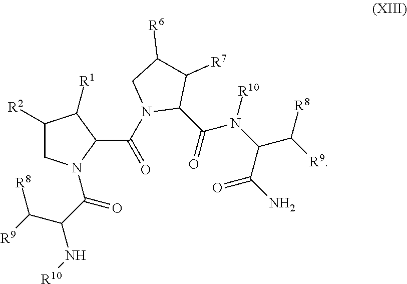

wherein:

R¹, R², R³, R⁴, R⁵, R⁶, R⁷, R⁸, R⁹, R¹⁰, R¹¹, and R¹² are as defined below. In some embodiments, step (a) is carried out at a temperature between about 15° C. and about 30° C. In some instances, step (b) is carried out at a temperature of between about −10° C. to about 30° C. In some embodiments, step (c) is carried out at a temperature between about 15° C. and about 30° C.

In some embodiments, the compound of Formula IX is produced by:
d) contacting a compound of Formula III:

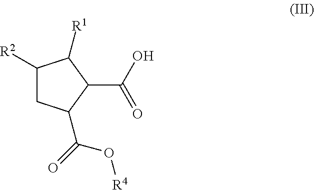

with an activating reagent and a compound of Formula II:

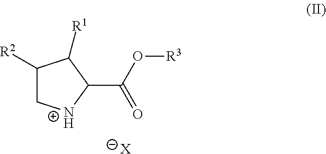

to produce a compound of Formula IV:

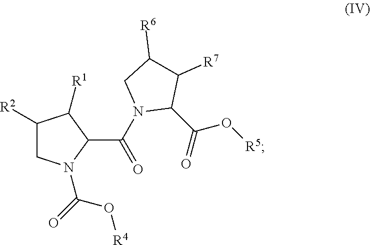

e) contacting the compound of Formula IV with a reagent capable of effecting hydrolysis to produce a compound of Formula V:

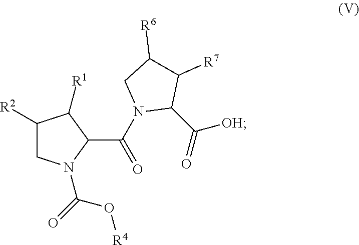

f) contacting the compound of Formula V with an activating reagent and a compound of Formula VIII:

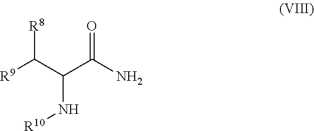

to produce a compound of Formula IX:

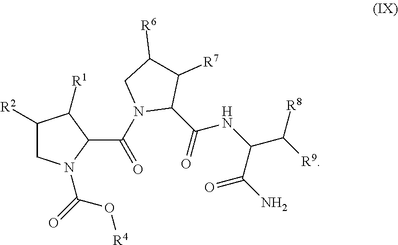

In some cases, step (e) is carried out at a temperature between about 15° C. and about 30° C. In some embodiments, step (f) is carried out at a temperature of between about 10° C. to about 30° C.

In some embodiments, the compound of Formula VIII is produced by the steps:
g) contacting a compound represented by Formula VI:

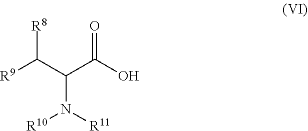

with an activating reagent to form a compound represented by Formula VII:

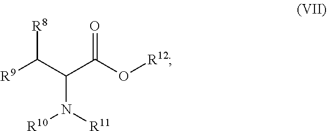

and
h) contacting the compound of Formula VII with an amine to produce the compound of Formula VIII. In some embodiments, step (g) is carried out at a temperature of between about 0° C. to 100° C. In some cases, step (h) is carried out at a temperature between about 15° C. to 30° C.

In some embodiments, the compound of Formula X is produced by contacting a compound of Formula VI:

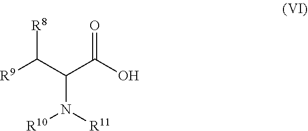

with an activated carbonyl compound. The process of claim 47 or 48, wherein producing the compound of Formula X is carried out at a temperature of between about 0° C. to about 30° C.

In some embodiments, the compound of Formula III is produced by contacting the compound of Formula II with an activated carbonyl reagent and a base. In some embodiments, the process further comprises contacting the compound of Formula VI with a base. In some instances, the base is NaHCO₃.

In some embodiments, the activating reagent comprises SOCl₂. In some instances, the alcohol is MeOH. In some embodiments, the activated carbonyl reagent is Cbz-Cl. In some cases, the base is a hydroxide salt. In some embodiments, the reagent capable of effecting hydrolysis comprises LiOH. For example, the reagent capable of effecting hydrolysis of the compound of Formula IV comprises LiOH. In some cases, the activating reagent comprises 1-ethyl-3-(3-dimethyllaminopropyl)carbodiimide. In some embodiments, the carbamate-cleaving reagent comprises palladium on carbon.

In some embodiments, the compound of Formula III is produced by contacting the compound of Formula I with an activating reagent and an alcohol to produce a reaction mixture comprising a compound of Formula II, and the reaction mixture is contacted with an activated carbonyl reagent and a base to produce the compound of Formula III.

In some embodiments, the compound of Formula VIII is produced by contacting the compound of Formula VI with an activating reagent and an alcohol to produce a reaction mixture comprising a compound of Formula VII, and the reaction mixture is contacted with an amine to produce the compound of Formula VIII. In some instances, the amine is NH₃.

In another aspect, a compound represented by the formula:

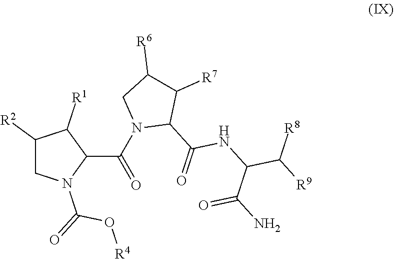

wherein:

R¹, R², R⁴, R⁶, R⁷, R⁸, and R⁹ are as defined below is provided.

In some embodiments, one or more of R¹, R², R⁶, and R⁷ is hydrogen. In some cases, R⁸ is methyl. In certain embodiments, R⁹ is hydroxyl. In some instances, R⁴ is benzyl.

In some embodiments, a compound represented by the formula:

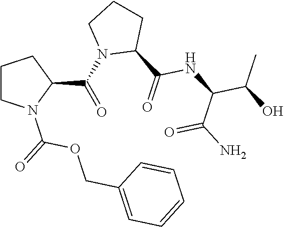

is provided.

In another aspect, a compound represented by the Formula X:

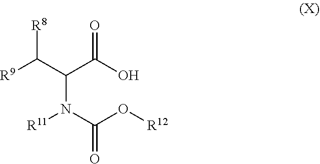

wherein:

R⁸, R⁹, R¹¹, and R¹² are as defined below is provided.

In some embodiments, R⁸ is methyl. In certain embodiments, R⁹ is hydroxyl. In some instances, R¹¹ is hydrogen. In some instances, R¹² is benzyl.

In some embodiments, a compound represented by the formula:

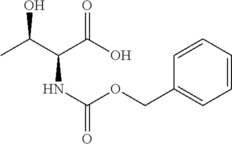 [SMILES: C[C@@H](O)[C@H](NC(=O)OCc1ccccc1)C(=O)O]

is provided.

### BRIEF DESCRIPTION OF DRAWINGS

FIG. 1 is a schematic of a six stage synthetic process for preparing intermediates KSM-1 of Formula IX and KSM-2 of Formula X used in production of GLYX-13, according to an embodiment; and

FIG. 2 is a schematic of a four stage synthetic process for preparing GLYX-13 from intermediates KSM-1 and KSM-2, according to an embodiment.

### DETAILED DESCRIPTION

Described herein is a new process for preparing dipyrrolidine peptide compounds. As a non-limiting example, the process may be used to prepare GLYX-13 or analogs or intermediates thereof. Advantageously, the process described herein may be used to prepare dipyrrolidine peptide compounds with higher purity and/or at less cost than known processes. Additionally, less toxic reagents and/or minimalist downstream processes may be used in contrast to known processes. Further, process may be scaled to produce industrial quantities of dipyrrolidine peptide compounds, e.g., greater than 1 kg of compound.

In some embodiments, the steps of the process may be carried out without using N-hydroxybenzotriazole (HOBT) and/or dichloromethane. This aspect may be advantageous since both HOBT and dichloromethane are costly raw materials, which increases the final process costs. Further, GLYX-13 is soluble in HOBT and the separation of this reaction mixture can be difficult. Consequently, the final purity of GLYX-13 may be compromised. Additionally, HOBT and dichloromethane are known to be toxic compounds, so their use introduces or increases the toxicity levels of the process. Of course, increased toxicity can result in increased process costs, for example, due to increased costs of handling toxic materials, increased waste disposal costs, and more expensive purification steps.

It will be appreciated by those of ordinary skill in the art that each of the embodiments contemplated herein may be utilized individually or combined in one or more manners different that the ones disclosed herein to produce an improved process for the production of dipyrrolidine peptide compounds. One skilled in the art will be able to select a suitable temperature and other such parameters in view of the reaction conditions being used in different embodiments.

### Processes

In one embodiment, a process is provided for preparing a compound of Formula XIII (pharmaceutically acceptable salts, stereoisomers, metabolites, and hydrates thereof):

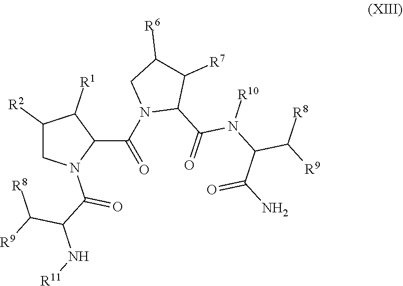

For example, a process is provided for preparing the compound GLYX-13. A disclosed process may include:
a) contacting a compound of Formula III:

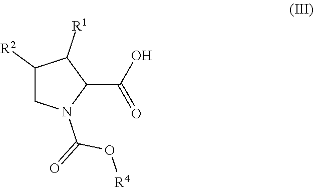

with an activating reagent and a compound of Formula II:

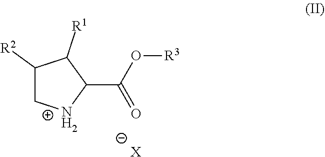

to produce a compound of Formula IV:

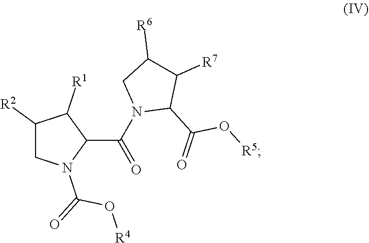

b) contacting the compound of Formula IV with a reagent capable of effecting hydrolysis to produce a compound of Formula V:

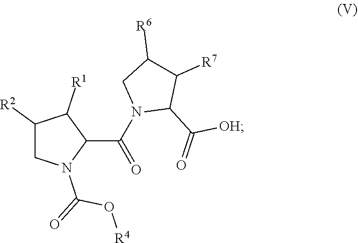

c) contacting the compound of Formula V with an activating reagent and a compound of Formula VIII:

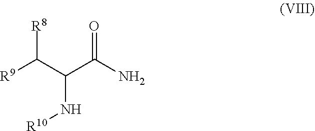

to produce a compound of Formula IX:

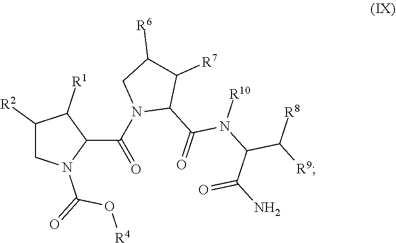

d) contacting the compound of Formula IX with a carbamate-cleaving reagent to produce a compound of Formula XI:

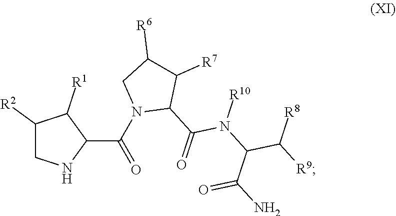

e) contacting a compound of Formula X:

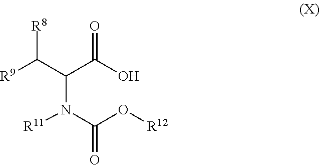

with an activating reagent and the compound of Formula XI to produce a compound of Formula XII:

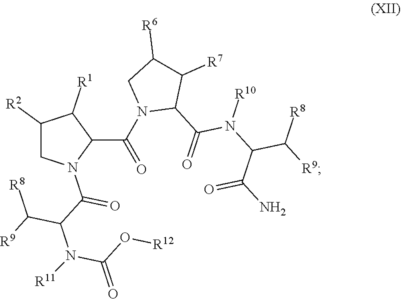

f) contacting the compound of Formula XII with a carbamate-cleaving reagent to produce a compound of Formula XIII:

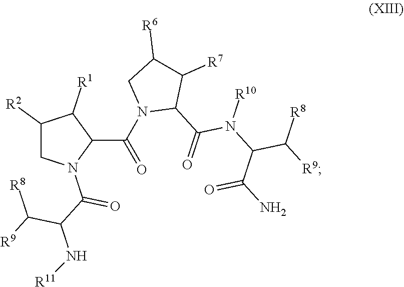

wherein:

R¹ and R² may be independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R¹ and R², together with the atoms to which they are attached, form a substituted or unsubstituted 4-6 membered heterocyclic or cycloalkyl ring;

R³ may be C₁₋₆alkyl optionally substituted by one or more substituents each independently selected from R^{f};

R⁴, R⁵, and R¹² may be independently —C₁₋₆alkylene-phenyl, wherein C₁₋₆alkylene is optionally substituted by one or more substituents each independently selected from R^{f};

R⁶ and R⁷ may be independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R⁶ and R⁷, together with the atoms to which they are attached, form a substituted or unsubstituted 4-6 membered heterocyclic or cycloalkyl ring;

R⁸ and R⁹ may be independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g};

R¹⁰ and R¹¹ are independently selected from the group consisting of hydrogen; C₁₋₆alkyl; —C(O)—C₁₋₆alkylene; —C(O)—O—C₁₋₆alkylene; and —C(O)-phenyl; wherein C₁₋₆alkyl, C₁₋₆alkylene, and phenyl are optionally independently substituted by one or more substituents selected from R^{a};

R^{b} may be selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₁₋₆alkoxy; C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;

R^{a} and R^{a′} may be selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;

R^{c} may be selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₁₋₆alkoxy; C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkyl C₃₋₆cycloalkylC₃₋₆cycloalkyl-C₁₋₆alkyl; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆ alkyl-carbonyl-N(R^{a})—;

R^{d} may be selected, independently for each occurrence, from the group consisting of C₁₋₆alkyl, C₁₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;

R^{e} may be selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;

R^{f} may be selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;

R^{g} may be selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—;

R^{a}R^{a′}N-carbonyl; R^{a}R^{a′}N—SO₂—; and C₁₋₄alkylS(O)_{w}—, where w is 0, 1, or 2; and

R^{x} may be selected, independently, from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alky 1 wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}.

In some embodiments, R¹ and R² may be hydrogen. In certain embodiments, R⁶ and R⁷ may be hydrogen. In some instances, R¹⁰ and/or R¹¹ may be hydrogen.

In some embodiments, at least one R⁸ may be hydrogen. In certain embodiments, at least one R⁸ may be methyl. At least one R⁹ may, in some embodiments, be hydroxyl. In certain instances, R⁸ may be methyl and R⁹ may be hydroxyl.

In certain embodiments, the compound of Formula IV may be

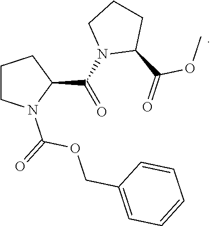

The compound of Formula V may be, for example,

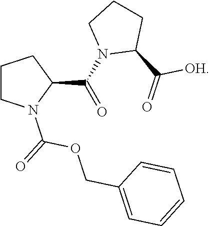

One non-limiting example of a compound of Formula VIII is

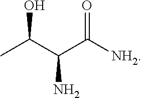

A compound of Formula IX may be exemplified by

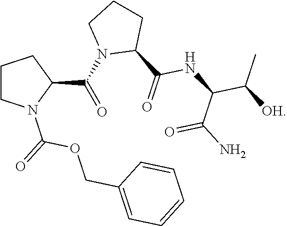

In some embodiments, a compound of Formula X may be

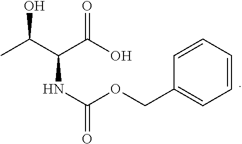 [SMILES: C[C@@H](O)[C@H](NC(=O)OCc1ccccc1)C(=O)O]

In some cases, a compound of Formula XI may be

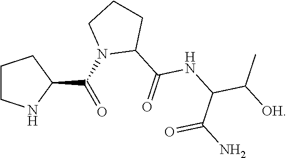

One non-limiting example of a compound of Formula XII is

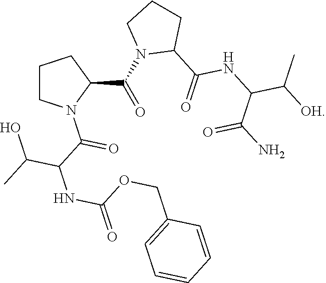

In some embodiments, the compound of Formula X may be produced by contacting a compound of Formula VI:

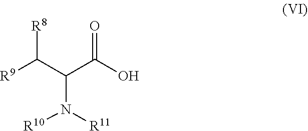

with an activated carbonyl compound. In certain embodiments, a base may be included in the reaction between the compound of the Formula VI and the activated carbonyl compound.

The compound of Formula VIII may be produced, in certain embodiments, by contacting a compound represented by Formula VI:

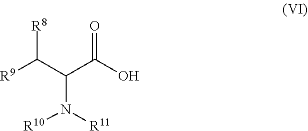

with an activating reagent to form a compound represented by Formula VII:

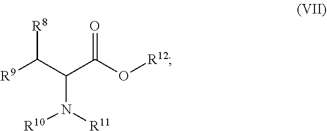

and
contacting the compound of Formula VII with an amine to produce the compound of Formula VIII. In some cases, the compound of Formula VIII may be produced by contacting the compound of Formula VI with an activating reagent and an alcohol to produce a reaction mixture comprising a compound of Formula VII, and the reaction mixture may be contacted with an amine to produce the compound of Formula VIII. For example, in such a process, the compound of Formula VII may not be isolated prior to reaction to form the compound of Formula VIII. However, in some embodiments, the compound of Formula VII may be isolated prior to reaction to form the compound of Formula III. Any suitable amine may be used. In some embodiments, the amine may be ammonia. In other embodiments, the amine may be a primary or secondary amine.

In some cases, the compound of Formula II may be produced by contacting a compound of Formula I:

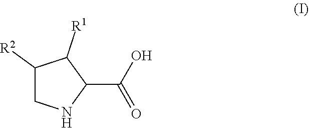

with an activating reagent and an alcohol. In some embodiments, the compound of Formula II may be a salt, where the counterion is represented by X⁻. The counterion may be any suitable ion. For example, the counterion may be a halide, e.g., fluoride, chloride, bromide, or iodide. In some embodiments, the compound of Formula I may be

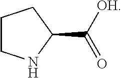

In certain embodiments, the compound represented by Formula II may be

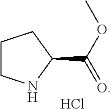

In certain embodiments, the compound of Formula III may be produced by contacting the compound of Formula II with an activated carbonyl reagent and a base. The compound of Formula II may be produced by contacting a compound of Formula I with an activating reagent and an alcohol. In some cases, the compound of Formula III may be produced by contacting the compound of Formula I with an activating reagent and an alcohol to produce a reaction mixture comprising a compound of Formula II, and the reaction mixture may be contacted with an activated carbonyl reagent and a base to produce the compound of Formula III. For example, in such a process, the compound of Formula II may not be isolated prior to reaction to form the compound of Formula III. In some embodiments, the compound of Formula II may be isolated prior to reaction to form the compound of Formula III. In certain embodiments, the compound of Formula III may be

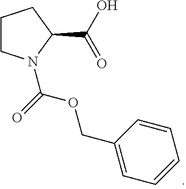 [SMILES: O=C(O)[C@@H]1CCCN1C(=O)OCc1ccccc1]

An activating agent may be any reagent capable of activating a carboxyl group for nucleophilic substitution. For example, in some embodiments, the activating agent may be used to convert the carboxyl group to an acyl halide, which may then undergo nucleophilic substitution. For instance, the reagent SOCl₂ may be used to convert the carboxyl group to an acyl chloride. In another embodiment, a carbodiimide may be used to activate a carboxyl group. For example, 1-ethyl-3-(3-dimethyllaminopropyl)carbodiimide (i.e., EDC), N,N′-dicyclohexylcarbodiimide (i.e., DCC), or N,N′-diisopropylcarbodiimide (i.e., DIC) may be used. In some embodiments, a carbodiimide-activated carboxyl group may be reacted to form an activated carbonyl group having more stability than a carbodiimide-activated carboxyl group. For example, the carbodiimide-activated carboxyl group may be reacted with N-hydroxysuccimide or a suitable alternative thereof to form a less labile activated carbonyl group.

An activated carbonyl compound may be reacted with a nucleophile to form, for example, an ester or amide. For example, in some embodiments, the activated carbonyl compound may be reacted with an alcohol (e.g., methanol, ethanol, or any other suitable alcohol) to form, for example, an ester or carbonate. In other embodiments, the activated carbonyl may be reacted with an amine to form, for example, an amide or carbamate. In one embodiment, the activated carbonyl compound may be a compound capable of forming a hydrogenation-labile carbonate or carbamate, e.g., benzyl chloroformate (i.e., Cbz-Cl).

In certain embodiments, reaction of an activated carbonyl compound with a nucleophile generates acid as a byproduct. For example, reaction of an acyl chloride with an alcohol or amine generates hydrochloric acid. In certain embodiments, it may be desirable to include a suitable acid scavenger in an acylation reaction. For example, a base such as a hydroxide salt (e.g., lithium hydroxide, sodium hydroxide, and the like), a carbonate (e.g., sodium carbonate, calcium carbonate, magnesium carbonate, and the like), or a bicarbonate (e.g., sodium bicarbonate) may be used.

A reagent capable of effecting hydrolysis may be any suitable reagent having this property. For example, the reagent may be a base such as a hydroxide salt (e.g., lithium hydroxide, sodium hydroxide, and the like).

A carbamate-cleaving reagent may be any suitable reagent capable of liberating an amine from a carbamate. The reagent may be chosen, for example, based on the identity of the carbamate. For instance, a base (e.g., a hydroxide salt) may be used to hydrolyze a carbamate. In embodiments where the carbamate comprises an alkyl-aryl ester (e.g., a benzyl ester), the carbamte-cleaving reagent may be a catalytic hydrogenation reagent (e.g., palladium on carbon (Pd/C)).

Each of the steps of the processes contemplated herein may be performed at any suitable temperature or gradient of temperatures. For example, a reaction may be carried out at a temperature of between about −20° C. to about 150° C., in some embodiments about 0° C. to about 100° C., in some embodiments between 15° C. and about 30° C., in some embodiments between about −10° C. to about 30° C., in some embodiments between about −20° C. to about 0° C., in some embodiments between about 0° C. to about 30° C., in some embodiments between about 0° C. to about 5° C., and in some embodiments between about 20° C. to about 30° C.

In certain embodiments, a lyophilization step may be included in the process. For example, the compound of Formula XIII may be lyophilized. Lyophilizing may be carried out at any suitable temperature or gradient of temperatures. For example, the lyophilization may be carried at a temperature of between about −50° C. to about 25° C. In some instances, the temperature may be increased from a first temperature of about −60° C. to about −40° C. to a second temperature of about 15° C. to about 30° C. The temperature gradient may occur over any suitable period of time. For example, in some embodiments, the period of time may be about 4 to about 48 hours, in some embodiments about 12 to about 36 hours, or in some embodiments about 20 to about 30 hours.

### Definitions

In some embodiments, the compounds, as described herein, may be substituted with any number of substituents or functional moieties. In general, the term “substituted” whether preceded by the term “optionally” or not, and substituents contained in formulas, refer to the replacement of hydrogen radicals in a given structure with the radical of a specified substituent.

In some instances, when more than one position in any given structure may be substituted with more than one substituent selected from a specified group, the substituent may be either the same or different at every position.

As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. In some embodiments, heteroatoms such as nitrogen may have hydrogen substituents and/or any permissible substituents of organic compounds described herein which satisfy the valencies of the heteroatoms. Non-limiting examples of substituents include acyl; aliphatic; heteroaliphatic; aryl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroarylthio; oxo; —F; —Cl; —Br; —I; —OH; —NO₂; —N₃; —CN; —SCN; —SR^{x}; —CF₃; —CH₂CF₃; —CHCl₂; —CH₂OH; —CH₂CH₂OH; —CH₂NH₂; —CH₂SO₂CH₃; —OR^{x}, —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein each occurrence of R^{x} independently includes, but is not limited to, hydrogen, halogen, acyl, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Furthermore, the compounds described herein are not intended to be limited in any manner by the permissible substituents of organic compounds. In some embodiments, combinations of substituents and variables described herein may be preferably those that result in the formation of stable compounds. The term “stable,” as used herein, refers to compounds which possess stability sufficient to allow manufacture and which maintain the integrity of the compound for a sufficient period of time to be detected and preferably for a sufficient period of time to be useful for the purposes detailed herein.

The term “acyl,” as used herein, refers to a moiety that includes a carbonyl group. In some embodiments, an acyl group may have a general formula selected from —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —NR^{x}C(O)R^{x}; —NR^{x}C(O)N(R^{x})₂; and —NR^{x}C(O)OR^{x}; wherein each occurrence of R^{x} independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles (e.g., morpholino, pyrrolidinyl, etc.), which may be optionally substituted with one or more functional groups or may be unsubstituted.

The terms “aryl” and “heteroaryl,” as used herein, refer to mono- or polycyclic unsaturated moieties having preferably 3-14 carbon atoms, each of which may be substituted or unsubstituted. In certain embodiments, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments, “heteroaryl” refers to a mono- or bicyclic heterocyclic ring system having one or two aromatic rings in which one, two, or three ring atoms are heteroatoms independently selected from the group consisting of S, O, and N and the remaining ring atoms are carbon. Non-limiting examples of heteroaryl groups include pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkenyloxy” used herein refers to a straight or branched alkenyl group attached to an oxygen (alkenyl-O). Exemplary alkenoxy groups include, but are not limited to, groups with an alkenyl group of 3-6 carbon atoms referred to herein as C₃₋₆alkenyloxy. Exemplary “alkenyloxy” groups include, but are not limited to allyloxy, butenyloxy, etc.

The term “alkoxy” as used herein refers to an alkyl group attached to an oxygen (—O— alkyl). Exemplary alkoxy groups include, but are not limited to, groups with an alkyl group of 1-12, 1-8, or 1-6 carbon atoms, referred to herein as C₁-C₁₂alkoxy, C₁₋₆alkoxy, and C₁-C₆alkoxy, respectively. Exemplary alkoxy groups include, but are not limited to methoxy, ethoxy, etc. Similarly, exemplary “alkenoxy” groups include, but are not limited to vinyloxy, allyloxy, butenoxy, etc.

The term “alkoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl, Exemplary alkoxycarbonyl groups include, but are not limited to, methoxycarbonyl, ethoxycarbonyl, t-butoxycarbonyl, etc.

The term “alkynyloxy” used herein refers to a straight or branched alkynyl group attached to an oxygen (alkynyl-O)). Exemplary alkynyloxy groups include, but are not limited to, propynyloxy.

The term “alkyl” as used herein refers to a saturated straight or branched hydrocarbon, for example, such as a straight or branched group of 1-6, 1-4, or 1-3 carbon atom, referred to herein as C₁-C₆alkyl, C₁-C₄alkyl, and C₁-C₃alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc. For example, alkyl may refer to a C₁₋₆ alkyl, optionally substituted by one, two, or three substituents selected from the group consisting of: halo, nitro, hydroxyl, —NH₂, —NH-alkyl, or alkoxy (e.g. —OCH₃).

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁-C₆alkyl carbonyl groups. Exemplary alkylcarbonyl groups include, but are not limited to, acetyl, propanoyl, isopropanoyl, butanoyl, etc.

The term “alkynyl” as used herein refers to an unsaturated straight or branched hydrocarbon having at least one carbon-carbon triple bond, such as a straight or branched group of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

Alkyl, alkenyl and alkynyl groups can optionally be substituted, if not indicated otherwise, with one or more groups selected from alkoxy, alkyl, cycloalkyl, amino, halogen, and —C(O)alkyl. In certain embodiments, the alkyl, alkenyl, and alkynyl groups are not substituted, i.e., they are unsubstituted.

The term “amide” or “amido” as used herein refers to a radical of the form —R^{a}C(O)N(R^{b})—, —R^{a}C(O)N(R^{b})R^{c}—, or —C(O)NR^{b}R^{c}, wherein R^{a}, R^{b}, and R^{c} are each independently selected from alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, and nitro. The amide can be attached to another group through the carbon, the nitrogen, R^{b}, R^{c}, or R^{a}. The amide also may be cyclic, for example R^{b} and R^{c}, R^{a} and R^{b}, or R^{a} and R^{c} may be joined to form a 3- to 12-membered ring, such as a 3- to 10-membered ring or a 5- to 6-membered ring. The term “carboxamido” refers to the structure —C(O)NR^{b}R^{c}.

The term “amine” or “amino” as used herein refers to a radical of the form —NR^{d}R^{e}, where R^{d} and R^{e} are independently selected from hydrogen, alkyl, alkenyl, alkynyl, aryl, arylalkyl, cycloalkyl, haloalkyl, heteroaryl, and heterocyclyl. The amino also may be cyclic, for example, R^{d} and R^{e} are joined together with the N to form a 3- to 12-membered ring, e.g., morpholino or piperidinyl. The term amino also includes the corresponding quaternary ammonium salt of any amino group, e.g., —[N(R^{d})(R^{e})(R^{f})]+. Exemplary amino groups include aminoalkyl groups, wherein at least one of R^{d}, R^{e}, or R^{f} is an alkyl group. In certain embodiment, R^{d} and R^{e} are hydrogen or alkyl.

The term “cycloalkoxy” as used herein refers to a cycloalkyl group attached to an oxygen (cycloalkyl-O—).

The term “cycloalkyl” as used herein refers to a monocyclic saturated or partially unsaturated hydrocarbon group of for example 3-6, or 4-6 carbons, referred to herein, e.g., as C₃₋₆cycloalkyl or C₄₋₆cycloalkyl and derived from a cycloalkane. Exemplary cycloalkyl groups include, but are not limited to, cyclohexyl, cyclohexenyl, cyclopentyl, cyclobutyl or, cyclopropyl.

The terms “halo” or “halogen” or “Hal” as used herein refer to F, Cl, Br, or I. The term “haloalkyl” as used herein refers to an alkyl group substituted with one or more halogen atoms.

The terms “heterocyclyl” or “heterocyclic group” are art-recognized and refer to saturated or partially unsaturated 3- to 10-membered ring structures, alternatively 3- to 7-membered rings, whose ring structures include one to four heteroatoms, such as nitrogen, oxygen, and sulfur. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isoxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. The heterocyclic ring may be substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. In certain embodiments, the heterocyclic group is not substituted, i.e., the heterocyclic group is unsubstituted.

The term “heteroaryloxy” refers to a heteroaryl-O— group.

The term “heterocycloalkyl” is art-recognized and refers to a saturated heterocyclyl group as defined above. The term “heterocyclylalkoxy” as used herein refers to a heterocyclyl attached to an alkoxy group. The term “heterocyclyloxyalkyl” refers to a heterocyclyl attached to an oxygen (—O—), which is attached to an alkyl group.

The term “heterocyclylalkoxy” as used herein refers to a heterocyclyl-alkyl-O-group.

The term “heterocyclyloxy” refers to a heterocyclyl-O— group.

The term “heterocyclyloxyalkyl” refers to a heterocyclyl-O-alkyl-group.

The terms “hydroxy” and “hydroxyl” as used herein refers to the radical —OH.

The term “oxo” as used herein refers to the radical ═O.

“Pharmaceutically or pharmacologically acceptable” include molecular entities and compositions that do not produce an adverse, allergic or other untoward reaction when administered to an animal, or a human, as appropriate. “For human administration, preparations should meet sterility, pyrogenicity, general safety and purity standards as required by FDA Office of Biologies standards.

As used in the present disclosure, the term “partial NMDA receptor agonist” is defined as a compound that is capable of binding to a glycine binding site of an NMDA receptor; at low concentrations a NMDA receptor agonist acts substantially as agonist and at high concentrations it acts substantially as an antagonist. These concentrations are experimentally determined for each and every “partial agonist.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein can exist in solvated as well as unsolvated forms with pharmaceutically acceptable solvents such as water, ethanol, and the like, and it is intended that the invention embrace both solvated and unsolvated forms. In one embodiment, the compound is amorphous. In one embodiment, the compound is a polymorph. In another embodiment, the compound is in a crystalline form.

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the e.g., Examples herein by substituting an isotopically labeled reagent for a non-isotopically labeled reagent.

As used in the present disclosure, “NMDA” is defined as N-methyl-D-aspartate.

### Examples

The following examples are provided for illustrative purposes only, and are not intended to limit the scope of the disclosure.

### Example 1: Synthesis of Intermediates

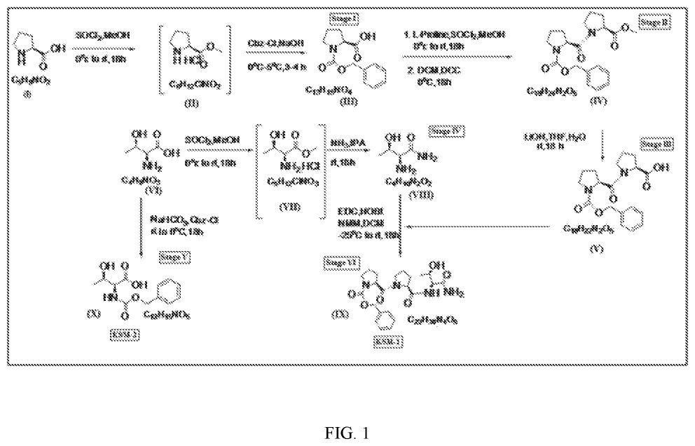

A chemical synthetic process for preparing KSM-1 and KSM-2 (identified below) using L-Proline (Compound I) and L-Threonine (Compound VI) as the starting materials is depicted in FIG. 1.

### Stage I—Preparation of (S)-1-(Benzyloxycarbonyl) pyrrolidine-2-carboxylic acid (Compound III)

Compound III was prepared using a two-step reaction. In the first step, L-Proline was reacted with SOCl₂ in the presence of methanol to produce Compound II, which was not isolated. In the second step, the reaction mixture from the first step containing Compound II was then converted to Compound III. The reaction was optimized and used to prepare Compound II in quantities of up to 25.0 kg in a production plant. Consistent purity (>95% by HPLC % AUC) was observed and yields were obtained in the range of 85% to 90%.

The reaction scheme is as follows:

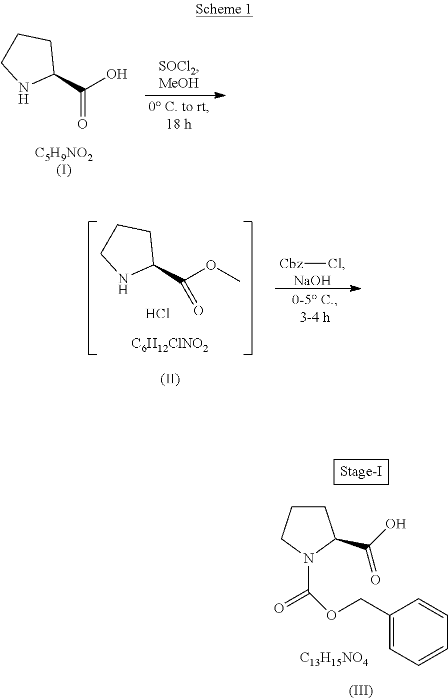

The reaction components used in this method can include those provided in Table 1:

TABLE 1

| S. No. | Name of the raw material | Qty | Units | MW | Moles | Molar Equivalents | |
|---|---|---|---|---|---|---|---|
| 1 | L-Proline | 5.00 | kg | 115.13 | 43.4 | 1.0 | |
| 2 | Thionyl chloride (Distilled) | 7.75 | kg | 119.0 | 65.1 | 1.5 | |
| 3 | Methanol Lot-I | 25.0 | L | — | — | 5.0 | Vol |
| 4 | Methanol Lot-II | 5.0 | L | — | — | 1.0 | Vol |
| 5 | Toluene Lot-I | 5.0 | L | — | — | 1.0 | Vol |
| 6 | Benzyl chloroformate (50% in | 11.37 | L | 170.60 | 47.7 | 1.1 | |
| | Toluene) | | | | | | |
| 7 | Sodium hydroxide (NaOH) | 6.94 | kg | 40.0 | 172.5 | 4.0 | |
| 8 | Water Lot-I | 35.0 | L | — | — | 7.0 | Vol |
| 9 | MTBE Lot-I | 20.0 | L | — | — | 4.0 | Vol |
| 10 | Toluene Lot-II | 20.0 | L | — | — | 4.0 | Vol |
| 11 | MTBE Lot-II | 15.0 | L | — | — | 3.0 | Vol |
| 12 | Concentrated HCl | 07.5 | L | — | — | 1.5 | Vol |
| 13 | Ethyl acetate Lot-I | 25.0 | L | — | — | 5.0 | Vol |
| 14 | Ethyl acetate Lot-II | 15.0 | L | — | — | 3.0 | Vol |
| 15 | Sodium chloride | 2.0 | kg | — | — | 0.4 | w/w |
| 16 | Water Lot-II | 20.0 | L | — | — | 4.0 | Vol |
| 17 | Sodium sulfate | 2.0 | kg | — | — | 0.4 | w/w |

Stage-I: methanol Lot-I was charged to the reactor at 20-30° C. L-Proline was added to the reactor at 20-30° C. The reaction mixture was cooled to 0-5° C. Distilled Thionyl chloride was added slowly to the reaction mixture at 0-5° C. The reaction mass temperature was raised to 20-25° C. and stirred for 12-18 h. Progress of the reaction was monitored by TLC. (Note: L-Proline should be less than 20%). Solvent was completely distilled from the reaction mass under reduced pressure at below 50° C. Methanol Lot-II was added and distilled under reduced pressure at 50° C. Toluene Lot-I was added and was distilled and degasified for 2 hours under reduced pressure at 50° C. The freshly prepared NaOH solution was added slowly to the reaction mass at below 20° C. (Note: NaOH Solution was prepared by dissolving NaOH in water Lot-I). The reaction mass was cooled to 0-5° C. and benzyl chloroformate was added slowly to the reaction mass at 0-5° C. and maintained at the same temperature for 3-4 hours. Progress of the reaction was monitored by TLC. (Note: the reaction intermediate Compound II (L-Proline Methyl ester) should be less than 2%). The reaction mass temperature was raised to 20-30° C. MTBE Lot-I was added to the reaction mass at 20-30° C. The reaction mass was stirred for 5-10 min and settled for 5-10 min. The aqueous layer was separated and washed with Toluene Lot-I, followed by MTBE Lot-II. The aqueous layer pH was adjusted to 1.0-2.0 with concentrated HCl. The reaction mass was stirred for 15 min and then ethyl acetate Lot-I was added. The organic layer was separated and the aqueous layer was extracted with ethyl acetate Lot-II. The organic layers were combined and washed with brine solution. (Note: The brine solution was prepared by adding sodium chloride to water Lot-II). The organic layer was dried over sodium sulfate. The organic layer was completely distilled under reduced pressure and degasified for 2 hours at below 50° C.

From the above reaction(s), 10.2 kg of Compound III were obtained with a yield of 94% and with a purity of 91.37%.

### Stage II—Preparation of (S)-Benzyl 2-((S)-2-(methoxycarbonyl) pyrrolidine-1-carbonyl)-pyrrolidine-1-carboxylate (Compound IV)

In this stage L-Proline of Formula I was reacted with SOCl₂ in presence of methanol to produce a compound of Formula II in a reaction mixture. The compound represented by Formula III obtained in stage 1 was then added to the reaction mixture, without isolating the compound of Formula II from the reaction mixture to produce a compound represented by Formula IV. This reaction was optimized and scaled up to 30.0 kg scale in the production plant and observed consistent quality. The HPLC purity is greater than 65% (AUC) and yields are in the range of 80% to 85%.

The reaction scheme involved in this method is as follows:

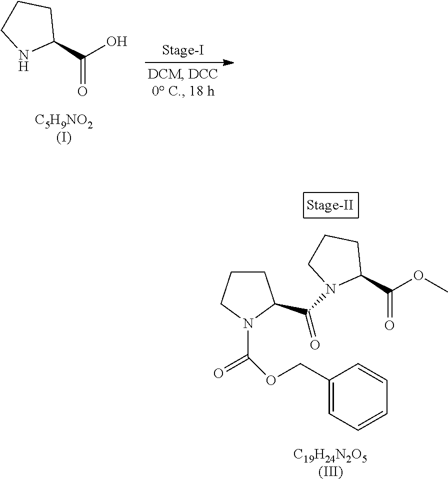

Raw materials used for this method are illustrated in Table 2 as follows:

TABLE 2

| S. No. | Name of the raw material | Qty | Units | MW | Moles | Molar Equivalents | |
|---|---|---|---|---|---|---|---|
| 1 | L-Proline | 3.00 | kg | 115.13 | 26.05 | 1.0 | |
| 2 | Thionyl chloride (Distilled) | 4.76 | kg | 119 | 39.08 | 1.5 | |
| 3 | Methanol Lot-I | 15.0 | L | — | — | 5.0 | Vol |
| 4 | Methanol Lot-II | 3.0 | L | — | — | 1.0 | Vol |
| 5 | Dichloromethane Lot-I | 24.0 | L | — | — | 8.0 | Vol |
| 5 | (S)-1-(benzyloxycarbonyl) | 5.199 | kg | 249.5 | 20.84 | 0.8 | |
| | pyrrolidine-2-carboxylic acid (Stage- | | | | | | |
| | I) (Formula III) | | | | | | |
| 6 | N,N-Dicyclohexylcarbodiimide | 6.448 | kg | 206.3 | 31.26 | 1.2 | |
| | (DCC) | | | | | | |
| 4 | Triethylamine | 2.63 | kg | 101 | 26.05 | 1.0 | |
| 5 | Dichloromethane Lot-II | 15.0 | L | — | — | 5 | Vol |
| 6 | Dichloromethane Lot-III | 15.0 | L | — | — | 5 | Vol |
| 7 | Ethyl acetate Lot-I | 24.0 | L | — | — | 8 | Vol |
| 8 | Ethyl acetate Lot-II | 12.0 | L | — | — | 4 | Vol |
| 9 | Sodium chloride Lot-I | 1.5 | kg | — | — | 0.5 | w/w |
| 10 | Water Lot-I | 15.0 | L | — | — | 5.0 | Vol |
| 11 | Sodium chloride Lot-II | 1.5 | kg | — | — | 0.5 | w/w |
| 12 | Water Lot-II | 15.0 | L | — | — | 5.0 | Vol |
| 13 | Citric acid | 0.624 | kg | — | — | 0.2 | w/w |
| 14 | Water Lot-III | 15.0 | L | — | — | 5.0 | Vol |
| 15 | Sodium bicarbonate | 1.5 | kg | — | — | 0.5 | w/w |
| 16 | Water Lot-IV | 15.0 | L | — | — | 5.0 | Vol |
| 17 | Sodium sulphate | 2.5 | kg | — | — | 0.83 | w/w |
| 18 | Ethyl acetate Lot-III | 3.0 | L | — | — | 1.0 | Vol |

In stage-II, methanol Lot-I was charged in to the reactor at 20-30° C. The compound represented by Formula I (L-Proline) was added to the reaction mass at 20-30° C. Reaction mass is cooled at 0-5° C. and thionyl chloride (Distilled) was added slowly to the reaction mass at 0-5° C. Then the reaction mixture was allowed raised to 20-35° C. and was maintained at 20-35° C. for 18 hours, to obtain the compound represented by Formula II. The progress of the reaction mixture was monitored by TLC for SM content. (Note: starting material should be less than 20%).

Reaction mass was distilled completely under reduced pressure at below 50° C. Methanol Lot-II was added and distilled under reduced pressure at below 50° C. and the reaction mass was cooled to 25-30° C. Dichloromethane Lot-I was added into the reactor at 25-30° C. Triethyl amine was added slowly to the reaction mixture at 0-10° C. Stage-I product, the compound represented by Formula III was dissolved in Diehloromethane Lot-II and the solution was added to the reaction mixture at below 20° C. and the reaction mixture was cooled to 0-5° C. The DCC solution was prepared by dissolving in Dichloromethane Lot-III and the solution was added slowly to the reaction mixture at 0-5° C., stirred for 4.0-4.5 hours. Reaction mass temperature was raised to 20-30° C. and stirred for 12-18 hours. Progress of the reaction was monitored by HPLC. (Note: Stage-I should be less than 2%). Solvent from the reaction mixture was distilled off completely under reduced pressure at below 45° C. and ethyl acetate Lot-I was added to the reaction mass. The reaction mass was cooled to 0-5° C. and stirred for 2-3 hours and reaction mass was filtered and washed with ethyl acetate Lot-II. (Note: By product DCU was filtered). All the organic layers were combined and washed with 2×15.0 L of brine solution. The organic layer was washed with 4% Citric acid solution and followed by sodium bicarbonate solution. (Note: Filtered the layers if any solids are observed in the layer). The organic layer was dried over sodium sulphate, filtered and washed the solid sodium sulphate with ethyl acetate Lot-III. Solvent was completely distilled under reduced pressure at below 50° C.

From the above reaction(s), 8.0 kg of compound represented by Formula IV was obtained with a yield of 85.2% and with a purity of 66.0% (HPLC AUC).

### Stage III—Preparation of Compound of (S)-1-((S)-1-(Benzyloxycarbonyl) pyrrolidine-2-carbonyl) pyrrolidine-2-carboxylic acid (Compound V)

The compound of Formula IV obtained above, was then reacted with LiOH, THF, water to produce a compound of Formula V. The reaction was optimized and performed up to 87.0 kg scale in the production plant and observed consistent quality (>95% by HPLC % PA) and yields (60%).

The reaction scheme involved in this method is as follows:

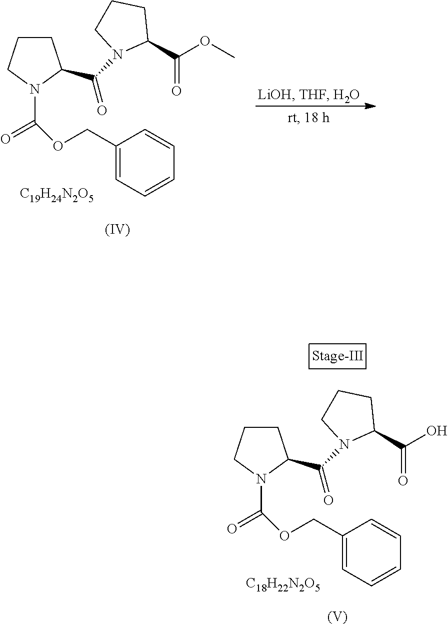

Raw materials used for this method are illustrated in Table 3 as follows:

TABLE 3

| S. No. | Name of the raw material | Qty | Units | MW | Moles | Molar Equivalents | |
|---|---|---|---|---|---|---|---|
| 1 | (S)-Benzyl 2-((S)-2-(methoxycarbonyl) | 6.0 | kg | 360.4 | 16.6 | 1.0 | |
| | pyrrolidine-1-carbonyl) pyrrolidine-1- | | | | | | |
| | carboxylate (Stage II)(Formula IV) | | | | | | |
| 2 | Lithium Hydroxide | 1.023 | kg | 41 | 24.9 | 1.5 | |
| 3 | THF | 30.0 | L | — | — | 5.0 | Vol |
| 4 | Water Lot-I | 30.0 | L | — | — | 5.0 | Vol |
| 5 | MTBE Lot-I | 12.0 | L | — | — | 2.0 | Vol |
| 6 | MTBE Lot-II | 12.0 | L | — | — | 2.0 | Vol |
| 7 | Conc. HCl | 4.5 | L | — | — | 0.75 | Vol |
| 8 | Water Lot-II | 15.0 | L | — | — | 2.5 | Vol |
| 9 | MTBE Lot-III | 12.0 | L | — | — | 2.0 | Vol |

In stage-III, THF and water Lot-I was charged into the reactor at 20-30° C. The Stage-II compound represented by Formula IV was added to the reaction mass at 20-30° C. Lithium Hydroxide was added to the reaction mass at 20-30° C. and reaction mass was stirred for 18 hours at 20-30° C. Progress of the reaction was monitored by TLC (Note: Stage-II should be less than 2%). Reaction mass was washed with MTBE twice Lot-1 and Lot-II and pH of aqueous layer was adjusted to 1.0-2.0 with concentrated HCl (sufficient quantity). (Note: Solid was precipitated during pH adjustment). Reaction mass was stirred for 1-1.5 hours at 20 to 30° C. and solid was filtered through Nutsche filter and washed with water Lot-II. Washed the cake with water Lot-III and MTBE Lot-III and dried the compound in HAD at 55-60° C.

From the above reaction(s), 3.42 kg of compound represented by Formula V was obtained with a yield of 59.0% and with a purity of 98.46%.

### Stage IV—Preparation of (2S, 3R)-2-Amino-3-hydroxybutanamide (Compound VIII)

In this stage the starting material L-Threonine of Formula VI was reacted with SOCl₂ in presence of methanol to produce a compound represented by Formula VII in a reaction mixture. The compound represented by Formula VII was further converted to a compound represented by Formula VIII without isolating the compound represented by Formula VII from the reaction mixture. The reaction was optimized and performed up to 5.0 kg scale in the production plant and observed consistent quality (>80% by HPLC % PA) and yields (65% to 70%).

The reaction scheme involved in this method is as follows:

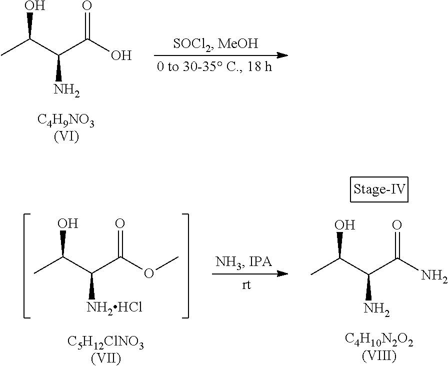

Raw materials used for this method are illustrated in Table 4 as follows:

TABLE 4

| S. No. | Name of the raw material | Qty | Units | MW | Moles | Molar Equivalents |
|---|---|---|---|---|---|---|
| 1 | L-Threonine | 5.00 | kg | 119.12 | 16.7 | 1.0 |
| 2 | Thionyl chloride (Distilled) | 7.45 | kg | 119 | 25.0 | 1.5 |
| 3 | Methanol Lot-I | 25.0 | L | — | — | 5.0 Vol |
| 5 | Methanol Lot-II | 5.0 | L | — | — | 1.0 Vol |
| 6 | Isopropanol Lot-I | 35.0 | L | — | — | 7.0 Vol |
| 7 | NH₃ gas | Q.S. | | — | — | |
| 8 | Isopropanol Lot-II | 10.0 | L | — | — | 2.0 Vol |
| 9 | MTBE Lot-I | 15.0 | L | — | — | 3.0 Vol |
| 9 | MTBE Lot-II | 5.0 | L | — | — | 1.0 Vol |

Stage-IV: methanol Lot-I was charged to the reactor at 20-30° C. A compound represented by Formula VI (L-threonine) was added to the reactor at 20-30° C. and the reaction mixture was cooled to 0-5° C. Distilled thionyl chloride was added slowly to the reaction mixture at 0-5° C. and temperature of reaction mass was raised to 20-25° C. and was maintained 18 hours to obtain a compound represented by Formula VII. Progress of the reaction was monitored by TLC. (Note: SM content should be less than 10%). Solvent from the reaction mass was completely distilled under reduced pressure at below 50° C. and methanol Lot-II was added and distilled under reduced pressure and degasified at below 50° C. for 2 hours. Isopropanol Lot-I was added to the reaction mass at 20-30° C. The resulting solution was charged into an autoclave at 20-30° C. and ammonia gas pressure to 4.5-5.0 Kg was applied to the reaction mass at 20-30° C. and maintained the pressure and temperature for 18 hours. (Note: Exotherm was observed during the ammonia pressurization.). Progress of the reaction was monitored by TLC. (Note: L-Threonine methyl ester should be less than 5%.). The reaction mass was filtered and washed with Isopropanol Lot-II and filtrate was distilled under reduced pressure at below 55° C. MTBE Lot-I was added slowly and stirred for 1 hour then filtered the solid and the solid was dried under HAD at 50-55° C.

From the above reaction(s), 3.0 kg of compound represented by Formula VIII was obtained with a yield of 69.7% and with a purity of 85.74%.

### Stage V-Preparation of (2S, 3R)-2-(Benzyloxycarbonylamino)-3-hydroxybutanoic acid (Compound X—KSM-2)

The starting material L-Threonine of Formula VI was reacted with NaHCO₃ and Cbz-Cl to produce KSM-2. The reaction was optimized and performed up to 10.0 kg scale in the production plant and observed consistent quality (>95% by HPLC % PA) and yields (45-50%).

The reaction scheme is as follows:

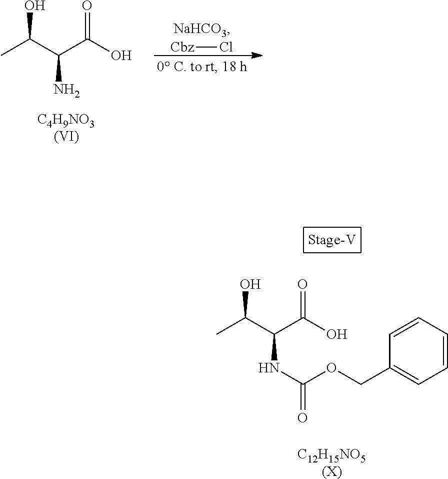

Raw materials used for this method are illustrated in Table 5 as follows:

TABLE 5

| S. No. | Name of the raw material | Qty | Units | MW | Moles | Molar Equivalents | |
|---|---|---|---|---|---|---|---|
| 1 | L-Threonine | 10.0 | kg | 119.12 | 83.89 | 1.0 | |
| 2 | Benzyl chloroformate (50% in Toluene) | 31.4 | L | 170.60 | 92.28 | 1.1 | |
| 3 | Sodium bicarbonate (NaHCO3) | 28.18 | kg | 84 | 335.56 | 4.0 | |
| 4 | Water Lot-I | 50.0 | L | — | — | 5.0 | Vol |
| 5 | MTBE Lot-I | 30.0 | L | — | — | 3.0 | Vol |
| 6 | Toluene Lot-I | 20.0 | L | — | — | 2.0 | Vol |
| 7 | MTBE Lot-II | 20.0 | L | — | — | 2.0 | Vol |
| 8 | Conc. HCl | 10.0 | L | — | — | 0.5 | Vol |
| 9 | Ethyl acetate Lot-I | 30.0 | L | — | — | 3.0 | Vol |
| 10 | Ethyl acetate Lot-II | 20.0 | L | — | — | 2.0 | Vol |
| 11 | Sodium chloride | 4.0 | kg | — | — | 0.4 | (w/w) |
| 12 | Water Lot-II | 20.0 | L | — | — | 2.0 | Vol |
| 13 | Sodium sulfate | 4.0 | kg | — | — | 0.4 | (w/w) |
| 14 | Ethyl acetate Lot-III | 100.0 | L | — | — | 10.0 | Vol |
| 15 | Dicyclohexaylamine (DCHA) | 30.42 | kg | 181.32 | 167.78 | 2.0 | |
| 16 | Ethyl acetate Lot-IV | 100.0 | L | — | — | 10.0 | Vol |
| 17 | Water Lot-III | 250.0 | L | — | — | 25.0 | Vol |
| 18 | Water Lot -IV | 50.0 | L | — | — | 5.0 | Vol |
| 19 | Sulphuric acid | 10.0 | L | — | — | 1.0 | Vol |
| 20 | Ethyl acetate Lot-V | 100.0 | L | — | — | 10.0 | Vol |
| 21 | Ethyl acetate Lot-VI | 100.0 | L | — | — | 10.0 | Vol |
| 22 | Sodium sulphate Lot-II | 4.0 | kg | — | — | 0.4 | times(w/w) |

In stage V, sodium bicarbonate and water Lot-I were charged into the reactor at 20 to 30° C. A compound of Formula VI (L-threonine) was added to the reaction mass at 20 to 30° C. and the reaction mass was cool to 0 to 5° C. Benzyl chloroformate was added to the reaction mass at 0 to 5° C. and the reaction mass was stirred at 0 to 5° C. for 1 hour. Temperature of reaction mass was cooled to 20 to 0° C. and was stirred at 20 to 30° C. for 18 hours. Progress of the reaction was monitored by TLC.

MTBE (Lot-I) was added to the reaction mass at 20-30° C. and reaction mass was stirred for 5-10 min, settled for 5-10 min, separated the layers. The aqueous layer washed with toluene Lot-I. The aqueous layer was washed with MTBE Lot-II and the pH of aqueous layer was adjusted to 1.0-2.0 with concentrated HCl. The reaction mass was stirred for 15 min, then ethyl acetate Lot-I was added. The organic layers were separated and again the aqueous layer was extracted with ethyl acetate Lot-II. The organic layers were combined and washed with brine solution. The organic layer dried with sodium sulfate and filtered. Ethyl acetate Lot-III was added to the organic layer. Dicyclohexylamine was added to the reaction mass at 20 to 30° C. and the reaction mass was stirred at 20 to 30° C., for 4 to 5 hours (Solid formation was observed). The reaction mass was cooled to 10 to 15° C. and maintained for 1 hour. Salt was filtered and washed with ethyl acetate Lot-IV. The wet salt was unloaded and charged into the reactor. Water Lot-III was added to reaction mass, and the pH was adjusted to 1.0-2.0 with 2N sulphuric acid. The reaction mass stirred for 15 min, ethyl acetate Lot-V was added in to reaction mass at 20 to 30° C. The layers were separated and again extracted the aqueous layer with ethyl acetate Lot-VI. The organic layer was combined and dried with sodium sulphate Lot-II and filtered. The organic layer was distilled out completely under vacuum at below 50° C. The liquid compound was unloaded in to HDPE container and samples were sent for complete QC analysis.

From the above reaction(s), 9.6 kg of KSM-2 was obtained with a yield of 45.0% and with a purity of 98.9%.

### Stage VI—Preparation of (S)-Benzyl 2-((S)-2-((2S, 3R)-1-amino-3-hydroxy-1-oxobutan-2-ylcarbamoyl) pyrrolidine-1-carbonyl) pyrrolidine-1-carboxylate (Compound IX—KSM-I)

The compound of Formula V obtained in stage III was coupled with the compound of Formula VIII obtained in stage VII to produce KSM-1. This reaction was optimized and scaled up to 6.0 kg scale in the production plant with consistent quality (>95% by HPLC % PA) and yields (45-50%).

The reaction scheme involved in this method is as follows:

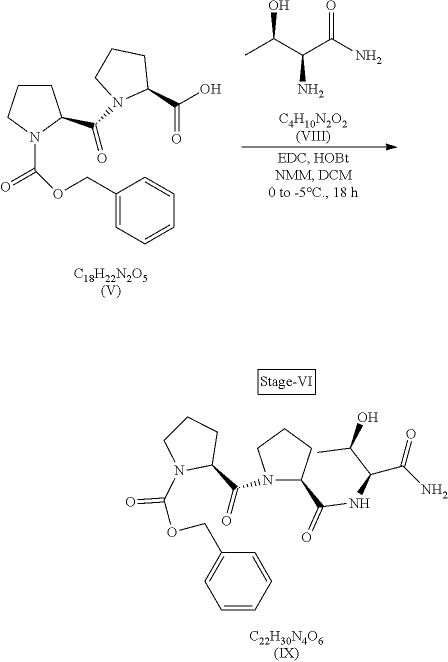

Raw materials used for this method are illustrated in Table 6 as follows:

TABLE 6

| S. No. | Name of the raw material | Qty | Units | MW | Moles | Molar Equivalents | |
|---|---|---|---|---|---|---|---|
| 1 | (S)-1-((S)-1-(Benzyloxycarbonyl) | 6.00 | kg | 346.36 | 0.0101 | 1.0 | |
| | pyrrolidine-2-carbonyl) pyrrolidine-2- | | | | | | |
| | carboxylic acid (Stage-III) (Formula V) | | | | | | |
| 2 | (2S,3R)-2-Amino-3-hydroxybutanamide | 3.26 | kg | 118.31 | 0.0163 | 1.6 | |
| | (Stage-IV) (Formula VIII) | | | | | | |
| 3 | 1-Hydroxybenzotriazole | 2.80 | kg | 135.10 | 0.0121 | 1.2 | |
| 4 | 1-(3-Dimethylaminopropyl)-3- | 3.90 | kg | 191.70 | 0.0121 | 1.2 | |
| | ethylcarbodiimide•HCl | | | | | | |
| 5 | N-Methyl morpholine | 4.38 | kg | 101.13 | 0.0252 | 2.5 | |
| 6 | Dichloromethane Lot-I | 60.0 | L | — | — | 10 | Vol |
| 7 | Dichloromethane Lot-II | 12.0 | L | — | — | 2 | Vol |
| 8 | Water Lot-I | 30.0 | L | — | — | 5 | Vol |
| 9 | Water Lot-II | 30.0 | L | — | — | 5 | Vol |
| 10 | Sodium chloride | 2.40 | kg | — | — | 0.4 | w/w |
| 11 | Water Lot-III | 24.0 | L | — | — | 4 | Vol |
| 12 | Dichloromethane Lot-III | 12.0 | L | — | — | 2 | Vol |
| 13 | Sodium sulphate | 3.00 | kg | — | — | 0.5 | w/w |
| 14 | Acetone | 3.60 | L | — | — | 0.6 | Vol |
| 15 | Methanol | 3.60 | L | — | — | 0.6 | Vol |
| 16 | n-Hexane Lot-I | 72.0 | L | — | — | 12 | Vol |
| 17 | n-Hexane Lot-I | 12.0 | L | — | — | 2 | Vol |
| 18 | Ethylacetete t-I | 12.0 | L | — | — | 2 | Vol |

In stage VI, dichloromethane and a compound represented by Formula V were charged into the reactor at 20-30° C. The reaction mass was cooled to −5 to 5° C. and 1-Hydroxybenzotriazole was added to the reaction mixture at −5 to 5° C. 1-(3-Dimethylaminopropyl)-3-ethylcarbodiimide HCl was added to the reaction mixture at −5 to 5° C. N-Methyl morpholine was slowly added to the reaction mixture at −5 to 5° C. and maintained for 30 minutes. A compound of Formula VIII dissolved in Dichloromethane Lot-II was added to the reaction mixture at −5 to 5° C. and maintained for 4 hours. The reaction mixture temperature was raised to 20-30° C. and maintained for 18 hours. Progress of the reaction was monitored by HPLC. (Note: SM (Stage-III) should be less than 5%). Water Lot-1 was charged into the reaction mass at 20-35° C. Separated the layers and again washed the organic layer with water Lot-II. Organic layers were combined and washed with brine solution. (Note: The brine solution was prepared by dissolving of sodium chloride in water Lot-III). The organic layer was filtered over celite bed and bed was washed with Dichloromethane Lot-III. The filtrate was dried over sodium sulphate and the solvent was distilled completely under reduced pressure at below 45° C. The crude was dissolved in Acetone and Methanol (1:1) mixture at 20 to 35° C. n-Hexane Lot-1 was added into the reaction mass at 20 to 35° C. and reaction mass was stirred for 4.0 hours at 20 to 35° C. Reaction mass was filtered through the Nutsche filter and washed with N-Hexane Lot-II. The compound was slurried with ethyl acetete and the compound was dried in a hot air drier at 45-50° C.

From the above reaction(s), 1.15 kg of KSM-1 was obtained with a yield of 24.0% and with a purity of 96.3%.

### Example 2: Synthesis of GLYX-13

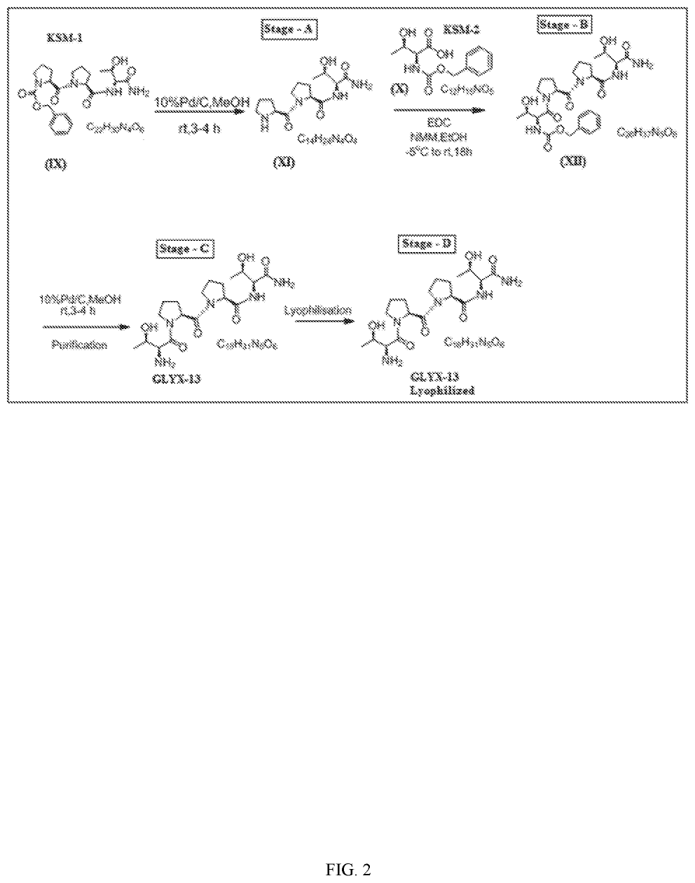

GLYX-13 was prepared as follows, using intermediates KSM-1 and KSM-2 produced in Example 1. The synthetic route for the same is provided in FIG. 2.

### Stage A—Preparation of(S)—N-((2S, 3R)-1-amino-3-hydroxy-1-oxobutan-2-yl)-1-((S)-pyrrolidine-2-carbonyl) pyrrolidine-2-carboxamide (Compound XI)

In this stage, KSM-1 was reacted with 10% Pd/C in presence of methanol to produce a compound represented by Formula XI. The reaction was optimized and performed up to 4.0 kg scale in the production plant and observed consistent quality (>80% by HPLC % PA) and yields (80% to 85%).

The reaction scheme involved in this method is as follows:

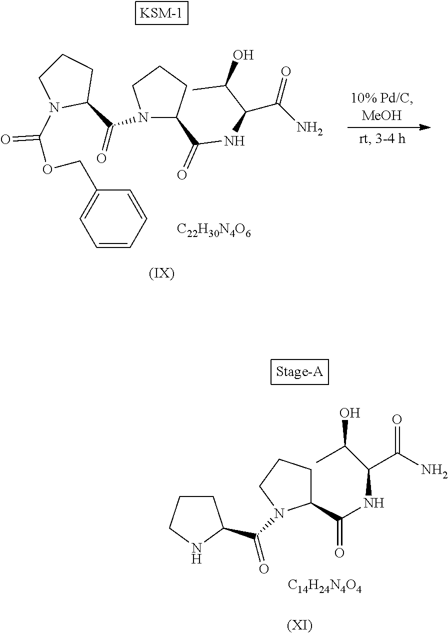

Raw materials used for this method are illustrated in Table 7 as follows:

TABLE 7

| S. No. | Name of the raw material | Qty | Units | MW | Moles | Molar Equivalents | |
|---|---|---|---|---|---|---|---|
| 1 | (S) - Benzyl 2-((S)-2-((2S,3R)-1- | 2.0 | kg | 446.5 | 4.4792 | 1.0 | |
| | amino-3-hydroxy-1-oxobutan-2- | | | | | | |
| | ylcarbamoyl) pyrrolidine-1- | | | | | | |
| | carbonyl) pyrrolidine-1-carboxylate | | | | | | |
| | (KSM-1) | | | | | | |
| 2 | 10% Pd/C | 0.4 | kg | — | — | 0.2 | times (w/w) |
| 3 | Methanol Lot-I | 80.0 | L | — | — | 40.0 | Vol |
| 4 | Methanol Lot-II | 13.2 | L | — | — | 6.6 | Vol |
| 5 | Methanol Lot-III | 5.2 | L | — | — | 2.64 | Vol |
| 6 | Hyflow | 2.0 | kg | — | — | 1.0 | times (w/w) |

| | | | | | | |
|---|---|---|---|---|---|---|
| 7 | Hydrogen gas | — | — | — | — | — |
| 8 | Nitrogen gas | — | — | — | — | — |

In stage A, 10% Palladium on Carbon (w/w, 50% wet) was charged into the pressure reactor at ambient temperature under nitrogen atmosphere. KSM-1 was dissolved in methanol in another container and sucked into above reactor under vacuum. Hydrogen pressure was maintained at 45-60 psi at ambient temperature for over a period of 5-6 hrs. Progress of the reaction mixture was monitored by HPLC for KSM-1 content; limit is not more than 5%. Hyflow bed was prepared with methanol (Lot-II). The reaction mass was filtered through nutsche filter under nitrogen atmosphere and bed was washed with Methanol Lot-III. Filtrate was transferred into the reactor and distilled completely under reduced pressure at below 50° C. (Bath temperature) to get the syrup and syrup material was unloaded into clean and dry container and samples were sent to QC for analysis.

From the above reaction(s), 1.31 kg of compound represented by Formula XI was obtained with a yield of 89.31% and with a purity of 93.63%.

### Stage B—Preparation of Benzyl (2S, 3R)-1-((S)-2-((S)-2-((2S, 3R)-1-amino-3-hydroxy-1-oxobutan-2-ylcarbamoyl) pyrrolidine-1-carbonyl) pyrrolidin-1-yl)-3-hydroxy-1-oxobutan-2-ylcarbamate (Compound XII)

In this stage the compound represented by Formula XI obtained above was reacted with KSM-2 to produce a compound represented by Formula XII. This reaction was optimized and scaled up to 3.0 kg scale in the production plant and obtained 25% to 28% yields with HPLC purity (>95%).

The reaction scheme is as follows:

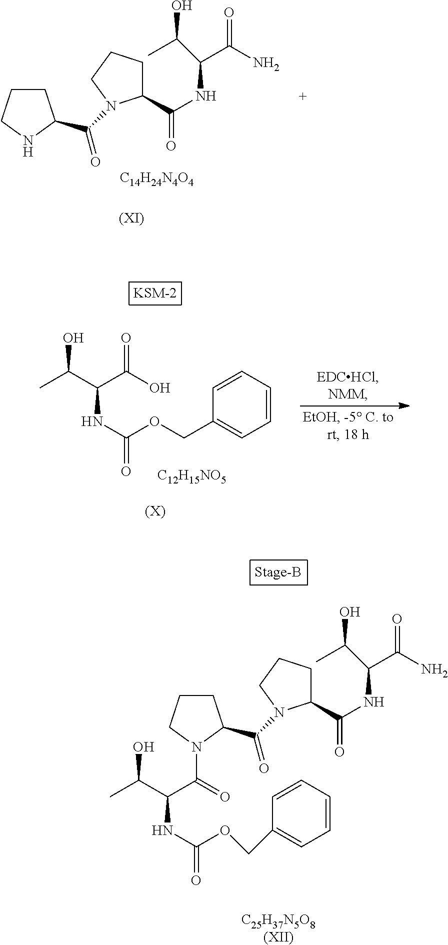

Raw materials used for this method are illustrated in Table 8 as follows:

TABLE 8

| S. No. | Name of the raw material | Qty | Units | MW | Moles | Molar Equivalents | |
|---|---|---|---|---|---|---|---|
| 1 | (S)-N-((2S,3R)-1-amino-3-hydroxy-1- | 1.30 | kg | 312.36 | 4.16 | 1.0 | |
| | oxobutan-2-yl)-1-((S)-pyrrolidine-2- | | | | | | |
| | carbonyl)pyrrolidine-2-carboxamide | | | | | | |
| | (Stage A) | | | | | | |
| 2 | Ethanol | 13.0 | L | — | — | 10.0 | Vol |
| 3 | 1-Ethyl-3-(3-Dimethylaminopropyl) | 957 | g | 191.7 | 4.99 | 1.2 | |
| | carbodiimide (EDC•HCl) | | | | | | |
| 4 | N-Methylmorpholine (NMM) | 767 | g | 101.15 | 7.58 | 1.82 | |
| 5 | (2S,3R)-2-(benzyloxycarbonylamino)-3- | 1.26 | kg | 253.25 | 4.99 | 1.2 | |
| | hydroxybutanoic acid (KSM-2) | | | | | | |
| 6 | Water Lot-1 | 5.2 | L | — | — | 4.0 | Vol |
| 7 | Dichloromethane Lot-1 | 5.85 | L | — | — | 4.5 | Vol |
| 8 | Isopropyl alcohol Lot-1 | 650 | mL | — | — | 0.5 | Vol |
| 9 | Dichloromethane Lot-2 | 5.85 | L | — | — | 4.5 | Vol |
| 10 | Isopropyl alcohol Lot-2 | 650 | mL | — | — | 0.5 | Vol |
| 11 | Dichloromethane Lot-3 | 5.85 | L | — | — | 4.5 | Vol |
| 12 | Isopropyl alcohol Lot-3 | 650 | mL | — | — | 0.5 | Vol |
| 13 | Dichloromethane Lot-4 | 5.85 | L | — | — | 4.5 | Vol |
| 14 | Isopropyl alcohol Lot-4 | 650 | mL | — | — | 0.5 | Vol |
| 15 | Potassium hydrogen sulfate Lot-1 | 650 | g | — | — | 0.5 | times w/w |
| 16 | Water Lot-2 | 1.30 | L | — | — | 1.0 | Vol |
| 17 | Potassium hydrogen sulfate Lot-2 | 650 | g | — | — | 0.5 | times w/w |
| 18 | Water Lot-3 | 1.30 | L | — | — | 1.0 | Vol |
| 19 | Sodium Sulfate | 1.30 | kg | — | — | 1.0 | Vol w/w |
| 20 | Silica Gel 230-400 Lot-1 | 1.3 | kg | — | — | 1.0 | Vol w/w |
| 21 | Silica Gel 230-400 Lot-1 | — | — | — | — | 10 | Vol w/w |
| 22 | Methanol Lot-1 | 91 | L | — | — | 70.0 | Vol |
| 23 | Dichloromethane Lot-4 | 910 | L | — | — | 700.0 | Vol |
| 24 | Methyl tert-butyl ether Lot-1 | 13 | L | — | — | 10.0 | Vol |
| 25 | Methyl tert-butyl ether Lot-2 | 2.6 | L | — | — | 2.0 | Vol |

Stage B: ethanol was charged into the reactor at 20 to 35° C. Compound represented by Formula XI was charged into the reactor under stirring at 20 to 35° C. and reaction mass was cooled to −5 to 0° C. EDC.HCl was charged into the reaction mass at −5 to 0° C. and reaction mass, was maintained at −5 to 0° C. for 10-15 minutes. N-Methyl morpholine was added drop wise to the above reaction mass at −5 to 0° C. and reaction mass was maintained at −5 to 0° C. for 10-15 minutes.

KSM-2 was charged into the reactor under stirring at −5 to 0° C. and reaction mass was maintained at −5 to 0° C. for 3.00 to 4.00 hours. The temperature of the reaction mass was raised to 20 to 35° C. and was maintained at 20 to 35° C. for 12-15 hours under stirring. (Note: Monitor the reaction mass by HPLC for Stage A content after 12.0 hours and thereafter every 2.0 hours. The content of stage A should not be more than 2.0%). Ethanol was distilled out completely under vacuum at below 50° C. (Hot water temperature) and reaction mass was cooled to 20 to 35° C. Water Lot-1 was charged into the residue obtained followed by 10% DCM-Isopropyl alcohol (Mixture of Dichloromethane Lot-1 & Isopropyl alcohol Lot-1 prepared in a cleaned HDPE container) into the reaction mass at 20-35° C.

Both the layers were separated and the aqueous layer was charged into the reactor. 10% DCM-Isopropyl alcohol (Mixture of Diehloromethane Lot-2 & Isopropyl alcohol Lot-2 prepared in a cleaned HDPE container) was charged into the reaction mass at 20 to 35° C. Both the layers were separated and the aqueous layer was charged back into the reactor. 10% IDCM-isopropyl alcohol (Mixture of Dichloromethane Lot-3 & Isopropyl alcohol Lot-3 prepared in a cleaned HDPE container) was charged into the reaction mass at 20 to 35° C. Both the layers were separated and the aqueous layer was charged back into the reactor. 10% DCM-Isopropyl alcohol (Mixture of Dichloromethane Lot-4 & Isopropyl alcohol Lot-4 prepared in a cleaned HDPE container) was charged into the reaction mass at 20 to 35° C. and separated both the layers. The above organic layers were combined and potassium hydrogen sulfate solution (Prepare a solution in a HDPE container by dissolving Potassium hydrogen sulfate Lot-1 in water Lot-2) was charged into the reaction mass at 20 to 35° C. Separated both the layers and charged back organic layer into the reactor. Potassium hydrogen sulfate solution (Prepared a solution in a HDPE container by dissolving Potassium hydrogen sulfate Lot-2 in water Lot-3) was charged into the reaction mass at 20 to 35° C. Separated both the layers and the organic layer was dried over Sodium sulfate and distilled out the solvent completely under vacuum at below 45° C. (Hot water temperature).

The above crude was absorbed with silica gel (100-200 mesh) Lot-1 in dichloromethane. Prepared the column with silica gel (100-200 mesh) Lot-2, and washed the silica gel bed with from Dichloromethane Lot-5 and charged the adsorbed compound into the column. Eluted the column with 0-10% Methanol Lot-1 in Dichloromethane Lot-5 and analyzed fractions by HPLC. Solvent was distilled out completely under vacuum at below 45° C. (Hot water temperature). Methyl tert-butyl ether Lot-1 was charged and stirred for 30 min. The solid was filtered through the Nutsche filter and washed with Methyl tert-butyl ether Lot-2 and samples were sent to QC for complete analysis. (Note: If product quality was found to be less than 95%, column purification should be repeated).

From the above reaction(s), 0.575 kg of compound represented by Formula XII was obtained with a yield of 17% and with a purity of 96.28%.

### Stage C—Preparation of Benzyl (S)—N-((2S, 3R)-1-amino-3-hydroxy-1-oxobutan-2-yl)-1-((S)-1-((2R, 3R)-2-amino-3-hydroxybutanoyl) pyrrolidine-2 carbonyl) pyrrolidine-2-carboxamide (GLYX-13)

In this reaction step the compound of Formula XII obtained above was reacted with 10% Pd in presence of methanol to produce GLYX-13. This reaction was optimized and performed up to 2.8 kg scale in the production plant and got 40% to 45% of yields with HPLC purity >98%.

The reaction scheme involved in this method is as follows:

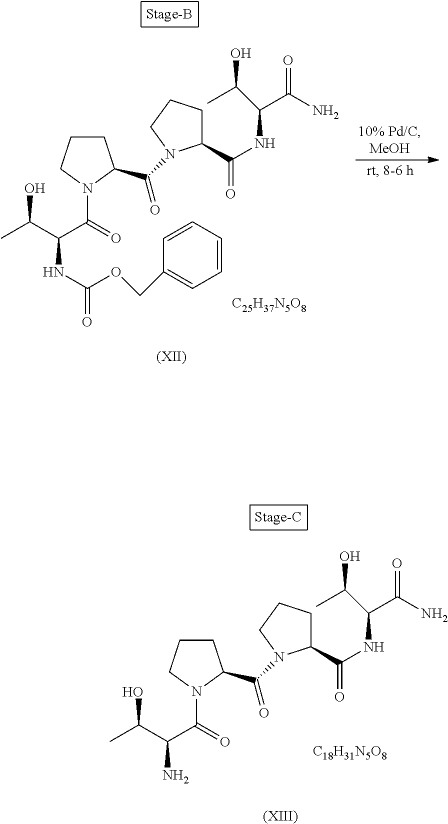

Raw materials used for this method are illustrated in Table 9 as follows:

TABLE 9

| S. No. | Name of the raw material | Qty | Units | MW | Moles | Molar Equivalents | |
|---|---|---|---|---|---|---|---|
| 1 | Benzyl (2S,3R)-1-((S)-2-((S)-2- | 2.8 | kg | 547.6 | 5.11 | 1.0 | |
| | ((2S,3R)-1-amino-3-hydroxy-1- | | | | | | |
| | oxobutan-2-ylcarbamoyl) | | | | | | |
| | pyrrolidine-1-carbonyl) | | | | | | |
| | pyrrolidin-1-yl)-3-hydroxy-1- | | | | | | |
| | oxobutan-2-ylcarbamate (Formula | | | | | | |
| | XII) (Stage-B) | | | | | | |
| 2 | 10% Pd/C (w/w, 50% wet) | 0.56 | kg | — | — | 0.2 | times (w/w) |
| 3 | Methanol Lot-I | 28 | L | — | — | 10.0 | Vol |
| 4 | Methanol Lot-II | 11.2 | L | — | — | 4.0 | Vol |
| 5 | Methanol Lot-III | 22.4 | L | — | — | 8.0 | Vol |
| 6 | Neutral Alumina Lot-I | 5.6 | kg | — | — | 2 | times (w/w) |
| 7 | Neutral Alumina Lot-2 | 1.0 | kg | — | — | 0.36 | times(w/w) |
| 8 | Neutral Alumina Lot-3 | 21.3 | kg | — | — | 7.64 | times(w/w) |
| 9 | Dichloromethane Lot-1 | 11.2 | L | — | — | 4 | Vol |
| 10 | Dichloromethane Lot-2 | 28 | L | — | — | 10 | Vol |
| 11 | Dichloromethane Lot-3 | 112 | L | — | — | 40 | Vol |
| 12 | Dichloromethane Lot-4 | 220 | L | — | — | 78.8 | Vol |
| 13 | Methanol Lot-4 | 0.56 | L | — | — | 0.2 | Vol |
| 14 | Dichloromethane Lot-5 | 220 | L | — | — | 78.8 | Vol |
| 15 | Methanol Lot-5 | 4.42 | L | — | — | 1.58 | Vol |
| 16 | Dichloromethane Lot-6 | 217 | L | — | — | 77.64 | Vol |
| 17 | Methanol Lot-6 | 6.72 | L | — | — | 2.4 | Vol |
| 18 | Dichloromethane Lot-7 | 107 | L | — | — | 38.2 | Vol |
| 19 | Methanol Lot-7 | 5.6 | L | — | — | 2.0 | Vol |
| 20 | Dichloromethane Lot-8 | 103 | L | — | — | 37.0 | Vol |
| 21 | Methanol Lot-8 | 8.8 | L | — | — | 3.17 | Vol |
| 22 | Dichloromethane Lot-9 | 100 | L | — | — | 35.8 | Vol |
| 23 | Dichloromethane Lot-10 | 20 | L | — | — | 7.17 | Vol |
| 24 | Methanol Lot-10 | 0.78 | L | — | — | 0.82 | Vol |
| 25 | Activated carbon (31 HW Neutral, | 0.47 | kg | — | — | 0.17 | Vol |
| | Mfr: Global Adsorbents Pvt Ltd.) | | | | | | |
| 26 | Hyflow Lot-2 | 2.8 | kg | — | — | 1.0 | times (w/w) |
| 27 | Methanol Lot-11 | 5.6 | L | — | — | 2.0 | Vol |
| 28 | Dichloromethane Lot-11 | 5.6 | L | — | — | 2.0 | Vol |
| 29 | Methanol Lot-12 | 1.12 | L | — | — | 0.4 | Vol |

| | | | | | | |
|---|---|---|---|---|---|---|
| 30 | Nitrogen cylinder | — | — | — | — | — |
| 31 | Hydrogen cylinder | — | — | — | — | — |

In an exemplary embodiment of stage C₁₋₁₀% Palladium Carbon (50% wet) was charged into the pressure reactor at ambient temperature under nitrogen atmosphere. Compound of Formula XII was dissolved in methanol in a separate container and sucked into the reactor under vacuum. Hydrogen pressure was maintained 45-60 psi at ambient temperature over a period of 6-8 hrs. Progress of the reaction was monitored by HPLC for stage-B (compound represented by Formula XII) content (limit is not more than 2%). If HPLC does not comply continue the stirring until it complies. Prepared the hyflow bed with methanol (Lot-II) and the reaction mass was filtered through hyflow bed under nitrogen atmosphere, and the filtrate was collected into a clean HDPE container. The bed was washed with Methanol Lot-III and the filtrate was transferred into the Rota Flask and distilled out the solvent completely under reduced pressure at below 50° C. (Bath temperature) to get the crude product. The material was unloaded into clean HDPE container under Nitrogen atmosphere.

Neutral Alumina Lot-1 was charged into the above HDPE container till uniform mixture was formed. The neutral Alumina bed was prepared with neutral alumina Lot-2 and dichloromethane Lot-1 in a glass column. The neutral Alumina Lot-3 was charged and Dichloromethane Lot-2 into the above prepared neutral Alumina bed. The adsorbed compound was charged into the column from op. no. 11. The column was eluted with Dichloromethane Lot-2 and collect 10 L fractions. The column was eluted with Dichloromethane Lot-3 and collected 10 L fractions. The column was eluted with Dichloromethane Lot-4 and Methanol Lot-4 (1%) and collected 10 L fractions. The column was eluted with Dichloromethane Lot-5 and Methanol Lot-5 (2%) and collected 10 L fractions. The column was eluted with Dichloromethane Lot-6 and Methanol Lot-6 (3%) and collected 10 L fractions. The column was eluted with Dichloromethane Lot-7 and Methanol Lot-7 (5%). and collected 10 L fractions. The column was eluted with Dichloromethane Lot-8 and Methanol Lot-8 (8%). and collected 10 L fractions. The column was eluted with Dichloromethane Lot-9 and Methanol Lot-9 (10%) and collected 10 L fractions. Fractions were analyzed by HPLC (above 97% purity and single max impurity >0.5% fractions are pooled together)

Ensured the reactor is clean and dry. The pure fractions were transferred into the reactor.

The solvent was distilled off completely under vacuum at below 45° C. (Hot water temperature). The material was cooled to 20 to 35° C. Charged Diehloromethane Lot-10 and Methanol Lot-10 into the material and stirred till dissolution. Activated carbon was charged into the above mixture at 20 to 35° C. and temperature was raised to 45 to 50° C.

Prepared the Hyflow bed with Hyflow Lot-2 and Methanol Lot-11 Filtered the reaction mass through the Hy-flow bed under nitrogen atmosphere and collect the filtrate into a clean HDPE container. Prepared solvent mixture with Dichloromethane Lot-11 and Methanol Lot-12 in a clean HDPE container and washed Nutsche filter with same solvent. Charged filtrate in to Rota evaporator and distilled out solvent under vacuum at below 50° C. Dry the compound in Rota evaporator for 5 to 6 hours at 50° C., send sample to QC for Methanol content (residual solvent) which should not be more than 3000 ppm. The material was cooled to 20 to 35° C. and the solid material was unloaded into clean and dry glass bottle. Samples were sent to QC for complete analysis.

From the above reaction(s), 0.92 kg of Glyx-13 was obtained with a yield of 43.5% and with a purity of 99.73%.

### Stage D—Lyophilization of GLYX-13

GLYX-13 obtained above was lyophilized and stored in amber colored bottles. This reaction was worked very well and performed up to 1.0 kg scale in the production plant successfully.

The reaction scheme involved in this method is as follows:

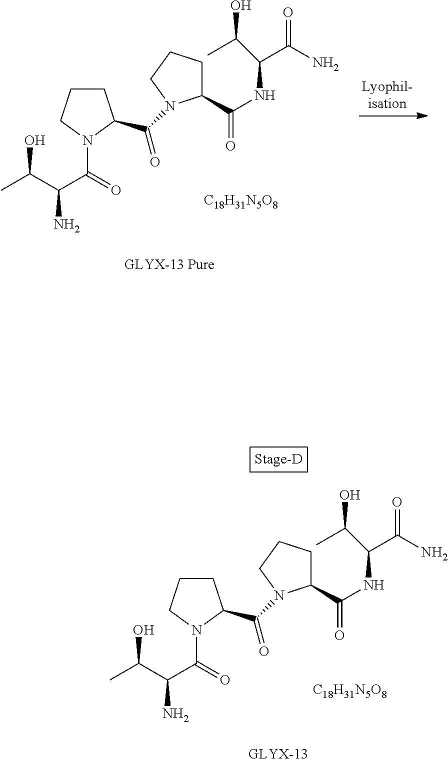

Raw materials used for this method are illustrated in Table 10 as follows: Table 10.

TABLE 10

| S. No. | Name of the raw material | Qty | Units | MW | Moles | Molar Equivalents |
|---|---|---|---|---|---|---|
| 1 | Benzyl (S)-N-((2S,3R)-1-amino- | 5 | g | 413.47 | 0.012 | 1 |
| | 3-hydroxy-1-oxobutan-2-yl)-1- | | | | | |
| | ((S)-1-((2R,3R)-2-amino-3- | | | | | |
| | hydroxybutanoyl) pyrrolidine-2 | | | | | |
| | carbonyl) pyrrolidine - 2- | | | | | |
| | carboxamide | | | | | |
| | (GLYX-13 Pure) (Stage C) | | | | | |
| 2 | Water (Milli-Q water) | 50 | ml | — | — | 10 Vol |
| 3 | Nitrogen | — | — | — | — | — |

In stage D, the GLYX-13 pure product was taken in the RBF with water (Milli-Q water) (10 Vol) and stirred for 30 minutes at 20-25° C. The solution was filtered through 0.22 micron filter paper, and the filtrate was taken in 100 ml RB flask and kept in the Lyophilizer and dried at −50 to +25° C. for 24 hours. The compound was placed into an Amber color glass bottle under Nitrogen atmosphere and closed with Teflon wad.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

What is claimed is:

1. A process for synthesizing a dipyrrolidine peptide compound or a pharmaceutically acceptable salt, stereoisomer, metabolite, or hydrate thereof, comprising the steps:
a) contacting a compound of Formula III: 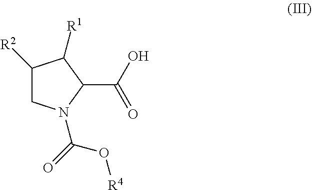
with an activating reagent and a compound of Formula II: 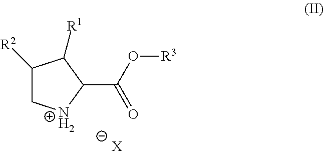
to produce a compound of Formula IV: 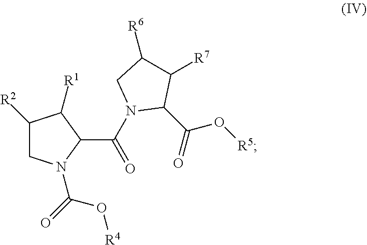
b) contacting the compound of Formula IV with a reagent capable of effecting hydrolysis to produce a compound of Formula V: 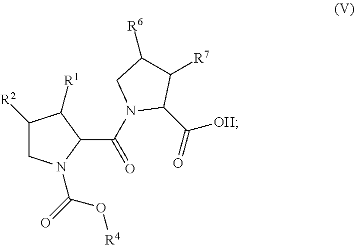
and
c) contacting the compound of Formula V with an activating reagent and a compound of Formula VIII: 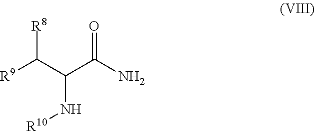
to produce a compound of Formula IX: 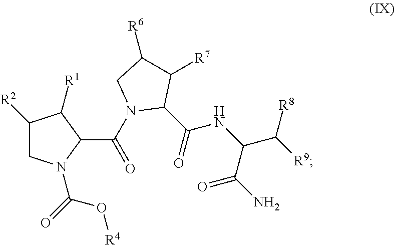
wherein:
R¹ and R² are independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R¹ and R², together with the atoms to which they are attached, form a substituted or unsubstituted 4-6 membered heterocyclic or cycloalkyl ring;
R³ is C₁₋₆alkyl optionally substituted by one or more substituents each independently selected from R^{f};
R⁴, R⁵, and R¹² are independently —C₁₋₆alkylene-phenyl, wherein C₁₋₆alkylene is optionally substituted by one or more substituents each independently selected from R^{f};
R⁶ and R⁷ are independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R⁶ and R⁷, together with the atoms to which they are attached, form a substituted or unsubstituted 4-6 membered heterocyclic or cycloalkyl ring;
R⁸ and R⁹ are independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g};
R¹⁰ and R¹¹ are independently selected from the group consisting of hydrogen; C₁₋₆alkyl; —C(O)—C₁₋₆alkylene; —C(O)—O—C₁₋₆alkylene; and —C(O)-phenyl; wherein C₁₋₆alkyl, C₁₋₆alkylene, and phenyl are optionally independently substituted by one or more substituents selected from R^{a};
R^{b} is selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₁₋₆alkoxy: C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{a} and R^{a′} are selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;
R^{c} is selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₁₋₆alkoxy; C₃₋₆alkenyloxy: C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{d} is selected, independently for each occurrence, from the group consisting of C₁₋₆alkyl, C₃₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;
R^{e} is selected, independently for each occurrence, from the group consisting of halogen; hydroxyl; —NO₂; —N₃; —CN; —SCN; C₁₋₄alkoxy; C₁₋alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{f} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{g} is selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2; and
R^{x} is selected, independently, from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alkyl-; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}.

2. The process of claim 1, further comprising the steps:
d) contacting the compound of Formula IX with a carbamate-cleaving reagent to produce a compound of Formula XI: 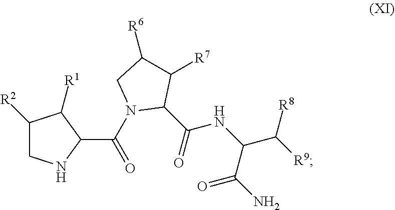
e) contacting a compound of Formula X: 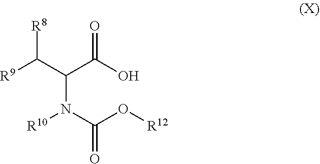
with an activating reagent and the compound of Formula XI to produce a compound of Formula XII: 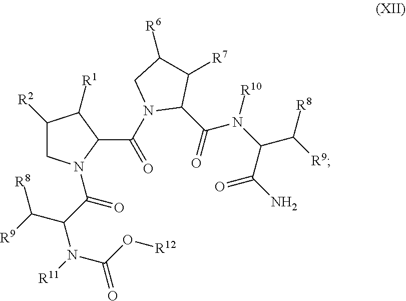
and
f) contacting the compound of Formula XII with a carbamate-cleaving reagent to produce a compound of Formula XIII:

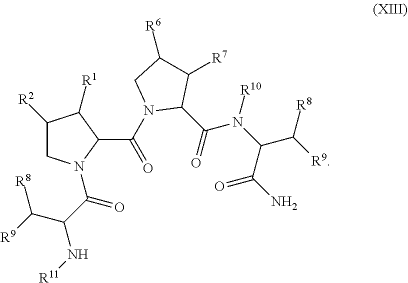

3. The process of claim 2, wherein the compound of Formula X is produced by contacting a compound of Formula VI: 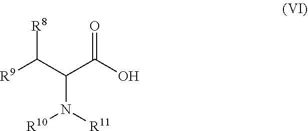
with an activated carbonyl compound.

4-19. (canceled)

20. The process of claim 2, wherein the activating reagent comprises 1-ethyl-3-(3-dimethyllaminopropyl)carbodiimide.

21. A process for preparing a dipyrrolidine peptide compound or a pharmaceutically acceptable salt, stereoisomer, metabolite, or hydrate thereof, comprising the steps:
a) contacting a compound of Formula IX: 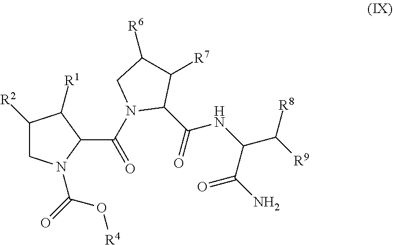
with a carbamate-cleaving reagent to produce a compound of Formula XI: 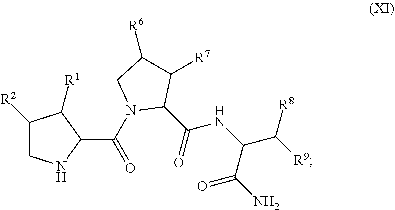
b) contacting a compound of Formula X: 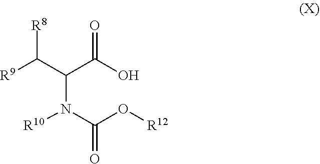
with an activating reagent and the compound of Formula XI in the presence of at least one solvent to produce a compound of Formula XII: 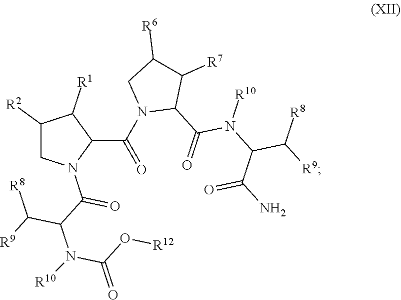
and
c) contacting the compound of Formula XII with a carbamate-cleaving reagent to produce a compound of Formula XIII: 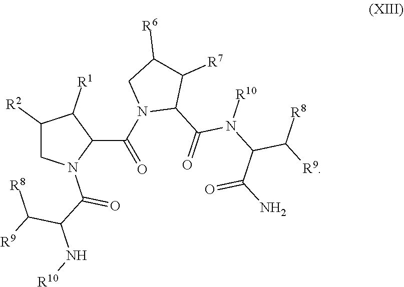
wherein:
R¹ and R² may be independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R¹ and R², together with the atoms to which they are attached, form a substituted or unsubstituted 4-6 membered heterocyclic or cycloalkyl ring;
R³ may be C₁₋₆alkyl optionally substituted by one or more substituents each independently selected from R^{f};
R⁴, R⁵, and R¹² may be independently —C₁₋₆alkylene-phenyl, wherein C₁₋₆alkylene is optionally substituted by one or more substituents each independently selected from R^{f};
R⁶ and R⁷ may be independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R⁶ and R⁷, together with the atoms to which they are attached, form a substituted or unsubstituted 4-6 membered heterocyclic or cycloalkyl ring;
R⁸ and R⁹ may be independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g};
R¹⁰ and R¹¹ are independently selected from the group consisting of hydrogen; C₁₋₆alkyl; —C(O)—C₁₋₆alkylene; —C(O)—O—C₁₋₆alkylene; and —C(O)-phenyl; wherein C₁₋₆alkyl, C₁₋₆alkylene, and phenyl are optionally independently substituted by one or more substituents selected from R^{a};
R^{b} may be selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₁₋₆alkoxy: C₃₋₆alkenyloxy: C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{a} and R^{a′} may be selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;
R^{c} may be selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₁₋₆alkoxy; C₃₋₆alkenyloxy: C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{d} may be selected, independently for each occurrence, from the group consisting of C₁₋₆alkyl, C₁₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;
R^{e} may be selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{f} may be selected, independently for each occurrence, from the group consisting of halogen; hydroxyl; —NO₂; —N₃; —CN; —SCN; C₁₋₄alkoxy; C₁₋₄alkoxy carbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{g} may be selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2; and
R^{x} may be selected, independently, from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alkyl-; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}.

22. The process of claim 21, wherein the carbamate-cleaving reagent comprises palladium on carbon.

23-26. (canceled)

27. The process of claim 21, wherein the compound of Formula IX is produced by:
d) contacting a compound of Formula III: 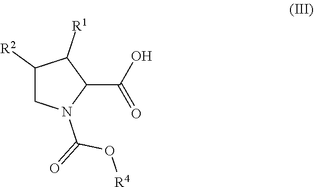
with an activating reagent and a compound of Formula II: 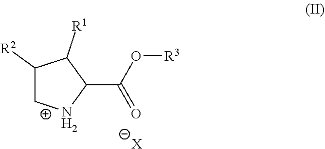
to produce a compound of Formula IV: 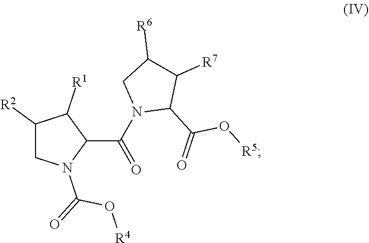
e) contacting the compound of Formula IV with a reagent capable of effecting hydrolysis to produce a compound of Formula V: 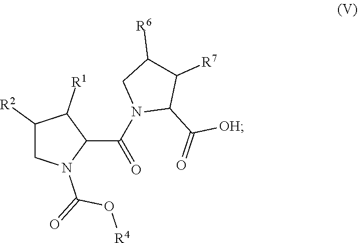
and
f) contacting the compound of Formula V with an activating reagent and a compound of Formula VIII: 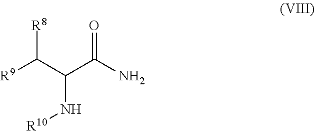
to produce a compound of Formula IX:

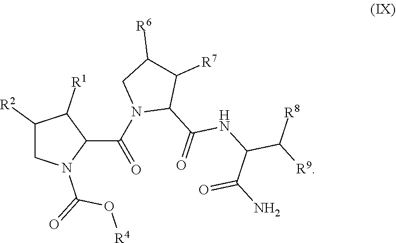

28. The process of claim 27, wherein the compound of Formula II is produced by contacting a compound of Formula I: 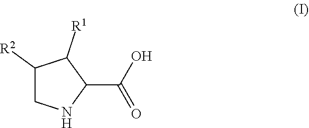
with an activating reagent and an alcohol.

29-46. (canceled)

47. The process of any one of claim 21, wherein the compound of Formula X is produced by contacting a compound of Formula VI: 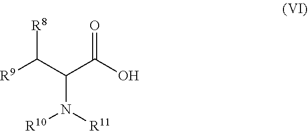
with an activated carbonyl compound.

48. The process of claim 47, wherein the activated carbonyl compound is Cbz-Cl.

49-62. (canceled)

63. A compound represented by the formula: 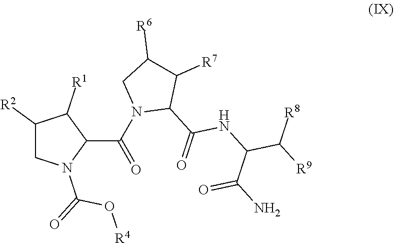
wherein:
R¹ and R² are independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R¹ and R², together with the atoms to which they are attached, form a substituted or unsubstituted 4-6 membered heterocyclic or cycloalkyl ring;
R⁴ is —C₁₋₆alkylene-phenyl, wherein C₁₋₆alkylene is optionally substituted by one or more substituents each independently selected from R^{f};
R⁶ and R⁷ are independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R⁶ and R⁷, together with the atoms to which they are attached, form a substituted or unsubstituted 4-6 membered heterocyclic or cycloalkyl ring;
R⁸ and R⁹ are independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g};
R^{b} is selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₁₋₆alkoxy; C₃₋₆alkenyloxy: C₃₋₆alkynyloxy: C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{a} and R^{a′} is selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;
R^{c} is selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₁₋₆alkoxy: C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{d} is selected, independently for each occurrence, from the group consisting of C₁₋₆alkyl, C₁₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;
R^{e} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{f} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{g} is selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2; and
R^{x} is selected, independently, from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alkyl-; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}.

64. The compound of claim 63, wherein one or more of R¹, R², R⁶, and R⁷ is hydrogen.

65-67. (canceled)

68. The compound of claim 63, represented by the formula:

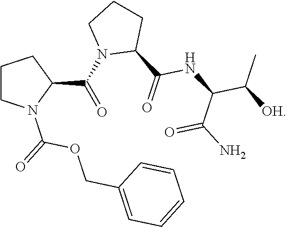

69. A compound represented by the Formula X: 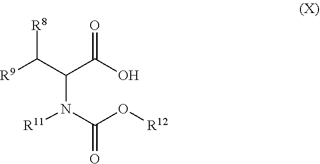
wherein:
R⁸ and R⁹ are independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g};
R¹¹ are independently selected from the group consisting of hydrogen; C₁₋₆alkyl; —C(O)—C₁₋₆alkylene; —C(O)—O—C₁₋₆alkylene; and —C(O)-phenyl; wherein C₁₋₆alkyl, C₁₋₆alkylene, and phenyl are optionally independently substituted by one or more substituents selected from R^{a};
R¹² is —C₁₋₆alkylene-phenyl, wherein C₁₋₆alkylene is optionally substituted by one or more substituents each independently selected from R^{f};
R^{b} is selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₁₋₆alkoxy: C₃₋₆alkenyloxy; C₃₋₆alkynyloxy: C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{a} and R^{a′} is selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;
R^{c} is selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₁₋₆alkoxy; C₃₋₆alkenyloxy: C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{d} is selected, independently for each occurrence, from the group consisting of C₁₋₆alkyl, C₃₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;
R^{e} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{f} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{g} is selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2; and
R^{x} is selected, independently, from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alkyl-; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}.

70. The compound of claim 69, wherein R⁸ is methyl.

71-73. (canceled)

74. The compound of claim 69, represented by the formula: 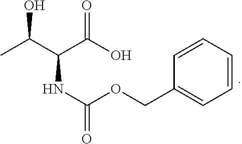 [SMILES: C[C@@H](O)[C@H](NC(=O)OCc1ccccc1)C(=O)O]
